SEQUENCE LISTING (6 sequences)

SEQ ID NO: 1 - protein, 219 aa, synthetic construct
  REGION 1..219: note = Synthetic construct
DIVMTQTPLSLSVTPGQPASISCKSSQSLLYSRGKTYLNWLLQKPGQSPQLLIYAVSKLDSGVPDRFSGSGSGTDFTLKISRVEAEDVGVYYCVQGTHYPFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 2 - protein, 444 aa, synthetic construct
  REGION 1..444: note = Synthetic construct
QVQLVQSGAEVKKPGSSVKVSCKASGYDFTRYYINWVRQAPGQGLEWMGWINPGSGNTKYNEKFKGRVTITADESTSTAYMELSSLRSEDTAVYYCAREGITVYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 3 - protein, 214 aa, synthetic construct
  REGION 1..214: note = Synthetic constuct
DIQMTQSPSTLSASVGDRVTITCRASQSLGNWLAWYQQKPGKAPKLLIYQASTLESGVPSRFSGSGSGTEFTLTISSLQPDDFATYYCQHYKGSFWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 4 - protein, 451 aa, synthetic construct
  REGION 1..451: note = Synthetic construct
EVQLLESGGGLVQPGGSLRLSCAASGFTFSSYPMSWVRQAPGKGLEWVSAISGSGGSTYYADSVKGRFTISRDNSKNTLYLQMNSLRAEDTAVYYCAREGGSGSYYNGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 5 - protein, 219 aa, synthetic construct
  REGION 1..219: note = Synthetic construct
DVVMTQSPLSLPVTLGQPASISCRSSQSLIYSDGNAYLHWFLQKPGQSPRLLIYKVSNRFSGVPDRFSGSGSGTDFTLKISRVEAEDVGVYYCSQSTHVPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 6 - protein, 442 aa, synthetic construct
  REGION 1..442: note = Synthetic construct
EVQLVESGGGLVQPGGSLRLSCAASGFTFSRYSMSWVRQAPGKGLELVAQINSVGNSTYYPDTVKGRFTISRDNAKNTLYLQMNSLRAEDTAVYYCASGDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

(19) United States
(12) Patent Application Publication
(10) Pub. No.: US 2025/0064417 A1
(43) Pub. Date: Feb. 27, 2025

(54) REGIONAL TAU IMAGING FOR DIAGNOSING AND TREATING ALZHEIMER'S DISEASE

(71) Applicant: Eli Lilly and Company, Indianapolis, IN (US)

(72) Inventors: Vikas KOTARI, Philadelphia, PA (US); Sergey SHCHERBININ, Indianapolis, IN (US); Sudeepti Suresh SOUTHEKAL, Basking Ridge, NJ (US); Ilke TUNALI, Indianapolis, IN (US)

(21) Appl. No.: 18/727,835

(22) PCT Filed: Jan. 30, 2023
(86) PCT No.: PCT/US2023/061544

§ 371 Date: Jul. 10, 2024

Related U.S. Application Data
(60) Provisional application No. 63/382,914, filed on Nov. 9, 2022.
(60) Provisional application No. 63/369,795, filed on Jul. 29, 2022.
(60) Provisional application No. 63/306,168, filed on Feb. 3, 2022.

(51) Int. Cl. A61B 6/50; A61B 6/00; A61B 6/03

(52) U.S. Cl. CPC A61B 6/501; A61B 6/037; A61B 6/5217

(57) ABSTRACT

Disclosed are methods for using regional tau PET scans for identifying a subject having or suspected of having, diagnosing, and treating Alzheimer's disease. The methods are particularly useful for treating and diagnosing a patient as susceptible and at risk for developing amyloid-beta and cognitive dysfunction using tau-PET imaging based on regional tau PET measures.

### BACKGROUND OF THE DISCLOSURE

The present disclosure relates generally to medicine. More particularly, the present disclosure is directed to identifying patients as having Alzheimer's disease or susceptible to developing Alzheimer's disease using tau PET imaging for diagnosis and/or treatment.

Alzheimer's disease (AD) is a progressive brain disorder that slowly destroys memory and thinking skills. AD is characterized by the formation of amyloid plaques, neurofibrillary tau tangles, and the loss of neuronal connections in the brain. There are five stages associated with Alzheimer's disease: preclinical Alzheimer's disease, Alzheimer's disease with mild cognitive impairment (AD with MCI), Alzheimer's disease with mild dementia, Alzheimer's disease with moderate dementia, and Alzheimer's disease with severe dementia. A subject can have preclinical Alzheimer's disease before the appearance of any symptoms. A subject with Alzheimer's disease with mild cognitive impairment presents as mild changes in memory and thinking ability. A subject with Alzheimer's disease with mild dementia experiences significant trouble with memory and thinking that impacts daily functioning. A subject with Alzheimer's disease with moderate dementia grows more confused and forgetful and begins needing more help with daily activities and self-care. At the stage of Alzheimer's disease with severe dementia, a subject may lose the ability to communicate coherently, may require daily assistance with personal care, and may experience a decline in physical abilities.

Diagnosis is generally based on the patient's history of the illness, cognitive testing, and medical imaging. Molecular imaging tracers for amyloid-beta and tau are used in Alzheimer's disease and other neurodegenerative disorders to determine the distribution of amyloid-beta and tau, respectively. While AD patients have both amyloid plaques and tau neurofibrillary tangles, the interaction of amyloid-β (Aβ) and tau resulting in cognitive impairment is not well understood. While amyloid pathology is present in AD, it is not consistently associated with severity of clinical features or disease duration (see, Villemagne et al., Ann Neurol. 2011; 69:181-192). One theory suggests that amyloid-β elevation induces downstream tau hyperphosphorylation and aggregation, synaptic dysfunction, and neuronal loss leading to cognitive impairment. Another theory suggests that Aβ and abnormal tau are two independent factors that exert synergistic effects on synaptic dysfunction and neuronal loss. It also remains challenging to determine if a patient with subtle cognitive symptoms suffers from preclinical AD that may progress to AD dementia. Early diagnosis and treatment may slow the clinical progression of AD as well as allowing for monitoring response to treatment.

Accordingly, there exists a continuing need to identify subjects having AD, stage disease progression in AD patients, and treat subjects with AD or susceptible to AD. A need also exists for improved methods for determining whether a subject is responding to therapies.

### BRIEF DESCRIPTION OF THE DRAWINGS

FIG. 1 depicts the percentage of amyloid positivity in the subjects from the A05 dataset (NCT02016560) using the PERSI reference.

FIG. 2 depicts the percentage of amyloid positivity in the subjects from the A05 dataset using the CereCrus reference.

FIG. 3 depicts the percentage of amyloid positivity in the subjects from the ADNI (Alzheimer's disease Neuroimaging Initiative) dataset using the CereCrus reference.

FIG. 4 depicts the SNR (signal-to-noise ratio) in the subjects from the A05dataset using the PERSI reference.

FIG. 5 depicts the SNR in the subjects from the A05 dataset using the CereCrus reference.

FIG. 6 depicts the SNR in the subjects from the ADNI (Alzheimer's disease Neuroimaging Initiative) dataset using the CereCrus reference.

FIG. 7 depicts the data from the A05 dataset for percentage of amyloid positivity in subjects, SNR in subjects, and correlations between regional SUVR and annual cognitive change, as described in Example 1, wherein r represents the Pearson correlation coefficient.

FIG. 8 depicts the data from the ADNI dataset for percentage of amyloid positivity in subjects, SNR in subjects, and correlations between regional SUVR and annual cognitive change, as described in Example 1, wherein r represents the Pearson correlation coefficient.

FIG. 9 depicts a scatter plot on the left showing AD_{signature} SUVR vs Eτ VOI SUVR with the subject's color coded based on amyloid status wherein Aβ+ is gray and Aβ− is black, and a zoomed in sub-section on the right highlighting subjects in the Eτ+/AD_{signature}− quadrant.

FIG. 10 depicts boxplots showing 18-month changes in (A) Eτ VOI SUVR, (B) AD_{signature} VOI SUVR, (C) cortical thickness, and (D) MMSE across quadrants, wherein Q1 corresponds to Eτ+/AD_{signature}+, Q2 corresponds to Eτ+/AD_{signature}−, and Q3 corresponds to Eτ−/AD_{signature}−.

FIG. 11 depicts a scatter plot on the left showing Braak stage vs AD_{signature} VOI SUVR, wherein the horizontal dotted line represents the AD_{signature} positivity cutoff of 1.1059, and a scatter plot on the right showing Braak state vs Eτ VOI SUVR, wherein the dotted line represents Eτ VOI cutoff of 1.1052.

### SUMMARY OF THE DISCLOSURE

The present disclosure relates generally to identifying patients having Alzheimer's disease and patients susceptible to developing Alzheimer's disease using tau imaging for diagnosis and treatment.

In one aspect, the present disclosure is directed to a method of treating a patient having or suspected of having Alzheimer's disease, the method comprising: analyzing a tau-PET scan of a brain region of the patient to determine a tau-PET SUVR; and administering an Alzheimer's disease therapy to the patient if the tau-PET SUVR ranges from about 1.10 to about 1.45.

In one aspect, the present disclosure is directed to a method of diagnosing a patient as having or suspected of having Alzheimer's disease, the method comprising: analyzing a tau-positron emission tomography (PET) scan from a brain region of the patient to determine a tau-PET SUVR; and diagnosing the patient as having or as suspected of having Alzheimer's disease if the tau-PET SUVR ranges from about 1.10 to about 1.45.

In one aspect, the present disclosure is directed to a method of identifying a patient as having or suspected of having Alzheimer's disease as a candidate patient for receiving an Alzheimer's disease therapy, the method comprising: analyzing a tau-positron emission tomography (PET) scan from a brain region of the patient to determine a tau-PET SUVR; and identifying the patient as a candidate patient for receiving an Alzheimer's disease therapy if the tau-PET SUVR ranges from about 1.10 to about 1.45.

In one aspect, the present disclosure is directed to a method of treating a patient having or suspected of having amyloid plaques, the method comprising: analyzing a tau image of a brain region of the patient to determine a tau-PET SUVR; and administering an Alzheimer's disease therapy to the patient if the tau-PET SUVR ranges from about 1.10 to about 1.45.

In one aspect, the present disclosure is directed to a method of diagnosing a patient as having or suspected of having amyloid plaques, the method comprising: analyzing a tau-positron emission tomography (PET) scan from a brain region of the patient to determine a tau-PET SUVR; and diagnosing the patient as having or as suspected of having amyloid plaques if the tau-PET SUVR ranges from about 1.10 to about 1.45.

In one aspect, the present disclosure is directed to a method of identifying a patient as having or suspected of having amyloid plaques as a candidate patient for receiving an Alzheimer's disease therapy, the method comprising: analyzing a tau-positron emission tomography (PET) scan from a brain region of the patient to determine a tau-PET SUVR; and identifying the patient as a candidate patient for receiving an Alzheimer's disease therapy if the tau-PET SUVR ranges from about 1.10 to about 1.45.

In one aspect, the present disclosure is directed to a method of identifying cognitive decline in a patient having or suspected of having Alzheimer's disease, the method comprising: analyzing a tau-positron emission tomography (PET) scan from a brain region of the patient to determine a tau-PET SUVR; and identifying the patient as having cognitive decline if the tau-PET SUVR ranges from about 1.10 to about 1.45.

In one aspect, the present disclosure is directed to a method of identifying whether a patient receiving an Alzheimer's disease therapy is responding to the Alzheimer's disease therapy, the method comprising: analyzing a tau-positron emission tomography (PET) scan from a brain region of the patient to determine a tau-PET SUVR; administering an Alzheimer's disease therapy to the patient; analyzing a tau-positron emission tomography (PET) scan from the brain region of the patient to determine a tau-PET SUVR after administering the Alzheimer's disease therapy; and identifying the patient as responding to the Alzheimer's disease therapy if the tau-PET SUVR before administering the Alzheimer's disease therapy is changed based on determination of the tau-PET SUVR after administering the Alzheimer's disease therapy.

In one aspect, the present disclosure is directed to a method of determining tau burden in a patient having or suspected of having Alzheimer's disease, the method comprising: analyzing a tau-positron emission tomography (PET) scan from a brain region of the patient to determine a tau-PET SUVR; and identifying the patient as having tau burden if the tau-PET SUVR is greater than 1.15.

In one aspect, the present disclosure is directed to a method for determining whether a patient is a candidate for enrollment in an Alzheimer's disease clinical trial, the method comprising: analyzing a tau-positron emission tomography (PET) scan from a brain region of the patient to determine a region-based tau-PET SUVR; and identifying the patient as a candidate for enrollment if the tau-PET SUVR is greater than 1.15.

### DETAILED DESCRIPTION

Unless defined otherwise, all technical and scientific terms used herein have the same meaning as commonly understood by one of ordinary skill in the art to which the disclosure belongs. Although any methods and materials similar to or equivalent to those described herein can be used in the practice or testing of the present disclosure, the preferred methods and materials are described below.

While the present disclosure is susceptible to various modifications and alternative forms, exemplary embodiments thereof are shown by way of example in the drawings and are herein described in detail. It should be understood, however, that the description of exemplary embodiments is not intended to limit the disclosure to the particular forms disclosed, but on the contrary, the intention is to cover all modifications, equivalents and alternatives falling within the scope of the disclosure as defined by the embodiments above and the claims below. Reference should therefore be made to the embodiments above and claims below for interpreting the scope of the present disclosure.

Unless defined otherwise, all technical and scientific terms used herein have the same meaning as commonly understood by one of skill in the art to which the invention pertains. Although any methods and materials similar to or equivalent to those described herein can be used in the practice or testing of the present disclosure, the preferred methods and materials are described herein. Moreover, reference to an element by the indefinite article “a” or “an” does not exclude the possibility that more than one element is present, unless the context clearly requires that there be one and only one element. The indefinite article “a” or “an” thus usually includes “at least one.” The term “about” means up to ±10%.

As used herein, “susceptible” and “at risk” refer to having little resistance to a certain disease, disorder or condition, including being genetically predisposed, having a family history of, and/or having symptoms of the disease, disorder or condition.

As used herein, a patient (also used interchangeably with “subject”) in need thereof, as it relates to the therapeutic uses herein, is one identified to require or desire medical intervention. Because some of the method embodiments of the present disclosure are directed to specific subsets or subclasses of identified patients (that is, the subset or subclass of patients “in need” of assistance in addressing one or more specific conditions noted herein), not all patients will fall within the subset or subclass of patients in need of treatment described herein. An effective amount is that amount of an agent necessary to inhibit the pathological diseases and disorders herein described. When at least one additional therapeutic agent is administered to a patient, such agents may be administered sequentially, concurrently, or simultaneously, in order to obtain the benefits of the agents. The term patient includes vertebrate animals, and preferably is a human patient.

In some embodiments, the biomarker F18-flortaucipir, which is a PET ligand, may be used for the purposes of the present disclosure. PET tau images can be, for example, quantitatively evaluated to estimate an SUVR (standardized uptake value ratio) by published methods (Pontecorvo et al., “A Multicentre Longitudinal Study of Flortaucipir (18F) in Normal Ageing, Mild Cognitive Impairment and Alzheimer's disease Dementia,” Brain 142:1723-35 (2019); Devous et al., “Test-Retest Reproducibility for the Tau PET Imaging Agent Flortaucipir F18,” Journal of Nuclear Medicine 59:937-43 (2018); Southekal et al., “Flortaucipir F18 Quantitation Using Parametric Estimation of Reference Signal Intensity,” J. Nucl. Med. 59:944-51 (2018), which are hereby incorporated by reference in their entireties) and/or to visually evaluate patients, e.g., to determine whether a patient has a pathological diagnosis of Alzheimer's disease (Fleisher et al., “Positron Emission Tomography Imaging With F18-flortaucipir and Postmortem Assessment of Alzheimer Disease Neuropathologic Changes,” JAMA Neurology 77:829-39 (2020), which is hereby incorporated by reference in its entirety). In the present embodiments, the SUVR was determined using methods similar to what was published by Pontecorvo et al. “Relationships between flortaucipir PET tau binding and amyloid burden, clinical diagnosis, age and cognition”, BRAIN 2017 doi:10.1093/brain/aww334 pages 1-16 (which paper is incorporated herein by reference). Lower SUVR values indicate less tau binding and amyloid-beta burden and slower AD progression while higher SUVR values indicate a higher tau and amyloid-beta burden and faster AD progression. In another embodiment, quantitative assessment by a flortaucipir scan is accomplished through an automated image processing pipeline as described in Southekal et al., “Flortaucipir F18 Quantitation Using Parametric Estimation of Reference Signal Intensity,” J. Nucl. Med. 59:944-951 (2018), which is hereby incorporated by reference in its entirety. In some embodiments, counts within a specific target region of interest in the brain (e.g., Automated Anatomical Labeling (AAL) and/or MUBADA, see Pontecorvo et al. “Relationships between flortaucipir PET tau binding and amyloid burden, clinical diagnosis, age and cognition”, BRAIN 2017 doi:10.1093/brain/aww334 pages 1-16; Devous et al, “Test-Retest Reproducibility for the Tau PET Imaging Agent Flortaucipir F18,” J. Nucl. Med. 59:937-943 (2018), which is hereby incorporated by reference in its entirety) are compared with a reference region wherein the reference region is, e.g., whole cerebellum, (wholeCere), cerebellar GM (cereCrus), atlas-based white matter (atlasWM), subject-specific WM (ssWM, e.g., using parametric estimate of reference signal intensity (PERSI), see Southekal et al., “Flortaucipir F18 Quantitation Using Parametric Estimation of Reference Signal Intensity,” J. Nucl. Med. 59:944-951 (2018), which is hereby incorporated by reference in its entirety). A preferred method of determining tau burden is a quantitative analysis reported as a standardized uptake value ratio (SUVR), which represents counts within a specific target region of interest in the brain (e.g., MUBADA and/or AAL brain regions) when compared with a reference region (e.g., cerebellar GM and/or PERSI).

As provided by the present disclosure, using region-based tau-PET SUVR, patients can be identified as having AD and/or susceptible to having AD. Patients identified as having AD and/or susceptible to having AD can be administered a treatment and the patient's response to treatment can be monitored using region-based tau-PET SUVR. As further provided by the present disclosure, using region-based tau-PET SUVR, patients can be identified as being amyloid-positive and/or susceptible to being amyloid-positive. Patients identified as amyloid-positive and/or susceptible to being amyloid-positive can be administered a treatment and the patient's response to treatment can be monitored using region-based tau-PET SUVR. In addition, as provided by the present disclosure, using region-based tau-PET SUVR, patients can also be identified as having AD cognitive decline and/or as susceptible to having AD cognitive decline. As further provided in the present disclosure, using region-based tau-PET SUVR, patients can be identified as candidate patients for therapy targeting amyloid plaque. Early identification of a patient using region-based tau-PET SUVR can permit earlier therapeutic intervention and prevent or delay disease progression. As provided by the present disclosure, using region-based tau-PET SUVR patients can be identified as candidates for clinical trial eligibility and for enrollment into customized AD trials.

In one aspect, the present disclosure is directed to a method of treating a patient having or suspected of having Alzheimer's disease. The method includes: analyzing a tau-PET scan of a brain region of the patient to determine a tau-PET SUVR; and administering an Alzheimer's disease therapy to the patient if the tau-PET SUVR ranges from about 1.10 to about 1.45.

Tau-positron emission tomography (tau-PET) is conducted by injection of suitable tau-PET tracers such as 18F-labeled arylquinoline derivatives and 11C-labeled phenyl/pyridinyl-butadienyl-benzothiazoles/benzothiazoliums, 5H-pyrio[4,3-b] indole. Particularly suitable tau-PET tracers include [18F]flortaucipir, [18F]RO948 (RO6958948), [11C]PBB3, [18F]MK-6240, [18F]PI-2620, [18F]GTP1, [18F]JNJ311 (JNJ64349311), [18F]JNJ067 (JNJ64326067), [18F]APN-1607 (PM-PBB3) and combinations thereof, (see, Bao et al., Aging Neurosci. 2021, 13:624330, which is incorporated by reference in its entirety). Any region of the brain is suitable for tau-PET analysis. Particularly suitable brain regions include central region, frontal lobe, temporal lobe, parietal lobe, occipital lobe, limbic lobe, insula, sub cortical gray nuclei (see, Tzourio-Mazoyer et al., NeuroImage, 2002, 15:273-289, which is incorporated by reference in its entirety). In some embodiments, the method further includes determining global tau-PET.

Tau-PET images are processed to obtain uniform image size and voxel dimensions. Standardized uptake value ratio (SUVR) images are produced using cerebellar gray matter and white matter as the reference regions. To determine low tau, intermediate tau, and high tau, white matter is particularly suitable for use as the reference region. SUVR is calculated using automated anatomical labeling atlas (AAL) or data driven volumes of interest for brain regions (see, Tzourio-Mazoyer et al., NeuroImage, 2002, 15:273-289, which is incorporated by reference in its entirety).

Amyloid-positron emission tomography (amyloid-PET) is conducted by injection of amyloid-PET tracers including [18F]florbetapir, carbon 11 (11C)-Pittsburgh Compound B (11C-PiB), [18F]florbetaben, [18F]NAV4694 (AZD4694), and [18F]flutemetamol, and combinations thereof, (as provided in Bao et al., Aging Neurosci. 2021, 13:624330, which is incorporated by reference in its entirety).

PET images are obtained using commercially available PET/computer tomography scanners such as Biograph mCT (Siemens), Biograph 6 Truepoint (Siemens), Discovery 690 (GE Healthcare).

Magnetic resonance imaging (MRI) is conducted by MRI images are obtained using commercially available scanners such as 3.0-T Discovery MR750 (GE Healthcare), 3.0-T Tim Trio (Siemens), 3.0-T Prisma (Siemens), and 1.5-T Magnetom® Avanto (Siemens).

Cortical thickness is measured by methods known in the art, such as MRI, for example.

The methods further include assessment of SUVR images using multiblock barycentric discriminant analysis (MUBADA) to identify brain regions that provide maximal diagnostic group differences. MUBADA was developed as a statistical method to predict group membership from large datasets that are structured into coherent blocks of variables (e.g., voxels), when the data have far more variables than participants (as is typically the case with neuroimaging data). MUBADA is an extension of mean-centered partial least-squares correlation, often used in neuroimaging research.

The methods further include assessment of medical history, neurological examination, and neuropsychological testing. This generally includes brain plaque imaging, mental or cognitive assessment and functional assessment. Suitable neuropsychological testing includes Clinical Dementia Rating-summary of boxes (CDR-SB), Mini-Mental State Examination (MMSE) and 13-item Alzheimer's disease Assessment Scale-Cognitive subscale (ADAS-Cog₁₃) score. MMSE is a diagnostic screening tool that measures cognitive abilities including, orientation to time and place, short term episodic memory, attention, problem solving, visuospatial abilities, and language and motor skills. ADAS-Cog₁₃ evaluates cognition and differentiates between normal cognitive functioning and impaired cognitive functioning where lower scores represent less impairment and higher scores represent more impairment. Functional assessment includes Alzheimer's disease Cooperative Study-Activities of Daily Living (ADCS-ADL). The cognitive and functional assessment can be used to determine changes in a patient's cognition (e.g., cognitive decline) and function (e.g., functional decline).

As used herein, “treating” (or “treat” or “treatment”) refers to processes involving a slowing, interrupting, arresting, controlling, stopping, reducing, or reversing the progression or severity of an existing symptom, disorder, condition, or disease, but does not necessarily involve a total elimination of all disease-related symptoms, conditions, or disorders associated with administration of the therapy. As used herein “prevent” refers to prophylactic administration of the therapy to an asymptomatic patient or a patient with pre-clinical Alzheimer's disease to stop onset or progression of the disease. Normal range of amyloid plaque is defined as demonstrating an amyloid plaque level of 25 centiloids or lower for two consecutive PET scans at least 6 months apart or a single PET scan demonstrating a plaque level of less than 11 centiloids. In the present disclosure, the term “normal range” of amyloid plaque in brain is used interchangeably with brain amyloid plaque is “cleared.” In some embodiments, the present disclosure results in about 15 to about 45 percent slowing of decline in the cognitive-functional composite endpoints from baseline over a duration of about 4 weeks, about 8 weeks, about 12 weeks, about 16 weeks, about 20 weeks, about 24 weeks, about 28 weeks, about 32 weeks, about 36 weeks, about 40 weeks, about 44 weeks, about 48 weeks, about 52 weeks, about 56 weeks, about 60 weeks, about 64 weeks, about 68 weeks, about 72 weeks, or 76 weeks. In some embodiments, the present disclosure results in about 15 to about 60 percent slowing of decline or disease progression on the Integrated Alzheimer's disease Rating Scale (iADRS) from baseline or as compared to untreated patient. Therapy is administered to the patient to slow disease progression. Disease progression is measured by mixed-model repeated-measures (MMRM) model, the Bayesian Disease Progression Model (DPM), Integrated Alzheimer's disease Rating Scale from baseline or compared to an untreated patient, Clinical Dementia Rating Scale-Sum of Boxes (CDR-SB) from baseline or compared to an untreated patient.

In some embodiments, treatment results in a decrease or reduction in amyloid deposits, amyloid-beta plaques, amyloid-beta load in the brain, and combinations thereof. In some embodiments, treatment results in a decrease or reduction in tau levels in the brain. In some embodiments, treatment results in a decrease, reduction, or prevent further increase of tau burden in specific regions of the brain. In some embodiments, treatment results in a decrease or reduction in tau levels in plasma. In some embodiments, such treatment results in decrease or reduction in neurofilament light chain (NfL) levels in brain of the patient having a disease characterized by Aβ plaques. In some embodiments, such treatment results in increase in Aβ42/40 ratio in plasma or cerebrospinal fluid (CSF) of the patient having a disease characterized by Aβ plaques. In some embodiments, such treatment results in decrease or reduction in glial fibrillary acidic protein (GFAP) in blood of the patient having a disease characterized by Aβ plaques. In some embodiments, such treatment results in decrease or reduction in P-tau 217 levels in a patient having a disease characterized by Aβ plaques.

Alzheimer's disease therapies include anti-amyloid antibodies, anti-tau antibodies, and combinations thereof. Suitable Alzheimer's disease therapies include anti-Ab 40 and 42 antibodies, N3pG targeting antibodies, OGAi (inhibitors of 3-O-(N-acetyl-D-glucosaminyl)-L- serine/threonine N-acetylglucosaminyl hydrolase, herein referred to as OGA) targeting antibodies and small molecules, tau targeting antibodies and small molecules. Some examples of these therapies include anti-N3pG antibodies (e.g., donanemab and N3pG IV or LY3372993,), anti-amyloid-beta antibodies (e.g., ADUHELM®, solanezumab, gantenerumab and lecanemab).

Donanemab is an IgG1 monoclonal antibody directed to the N-terminal pyroglutamate modification of the third amino acid of amyloid beta (N3pGlu Aβ) epitope that is present only in brain amyloid beta plaques. The mechanism of action of donanemab is the targeting and removal of existing amyloid plaque. The methods of making and using donanemab are described in U.S. Pat. Nos. 8,679,498 and 8,961,972 (referred to as antibody B12L therein), which are expressly incorporated herein by reference. Suitable dosing (as described in PCT/US2017/038999 and PCT/US2022/011894, which are expressly incorporated herein by reference) includes, among others, three doses of about 100 mg to about 700 mg every 4 weeks (i.e., at a frequency of once every four weeks) followed by a dose of about 700 mg to about 1400 mg every 4 weeks. The dosing regimen can be delivered for 1) up to 72 weeks, 2) until the patient is amyloid negative as measured by PET, 3) until amyloid is cleared as measured by PET, or 4) chronically. In some embodiments, the human subject is administered the anti-N3pGlu Aβ antibody (including, e.g., the first dose and/or the second dose) for a duration sufficient to bring the amyloid plaque in the subject's brain to a normal range (or until brain amyloid plaque is cleared). In the present disclosure, the term “normal range” of amyloid plaque in brain is used interchangeably with brain amyloid plaque is “cleared.” The normal range of amyloid plaque is defined as demonstrating an amyloid plaque level of 25 centiloids or lower for two consecutive PET scans at least 6 months apart or a single PET scan demonstrating a plaque level of less than 11 centiloids. In one embodiment, 700 mg of donanemab is administered every 4 weeks for first 3 doses, then administered at a dose of 1400 mg every 4 weeks until brain amyloid plaque is cleared. In some embodiments, the antibody has the following sequence:

Light Chain:
SEQ. ID NO. 1
DIVMTQTPLSLSVTPGQPASISCKSSQSLLYSRGKTYLNWLLQKPGQSP
QLLIYAVSKLDSGVPDRFSGSGSGTDFTLKISRVEAEDVGVYYCVQGTH
YPFTFGQGTKLEIKRTVAAPSVFIFPPSDEQLKSGTASVVCLLNNFYPR
EAKVQWKVDNALQSGNSQESVTEQDSKDSTYSLSSTLTLSKADYEKHKV
YACEVTHQGLSSPVTKSFNRGEC
Heavy Chain:
SEQ ID NO. 2
QVQLVQSGAEVKKPGSSVKVSCKASGYDFTRYYINWVRQAPGQGLEWMG
WINPGSGNTKYNEKFKGRVTITADESTSTAYMELSSLRSEDTAVYYCAR
EGITVYWGQGTTVTVSSASTKGPSVFPLAPSSKSTSGGTAALGCLVKDY
FPEPVTVSWNSGALTSGVHTFPAVLQSSGLYSLSSVVTVPSSSLGTQTY
ICNVNHKPSNTKVDKKVEPKSCDKTHTCPPCPAPELLGGPSVFLFPPKP
KDTLMISRTPEVTCVVVDVSHEDPEVKFNWYVDGVEVHNAKTKPREEQY
NSTYRVVSVLTVLHQDWLNGKEYKCKVSNKALPAPIEKTISKAKGQPRE
PQVYTLPPSRDELTKNQVSLTCLVKGFYPSDIAVEWESNGQPENNYKTT
PPVLDSDGSFFLYSKLTVDKSRWQQGNVFSCSVMHEALHNHYTQKSLSL
SPG

N3pG IV or LY3372993 is an IgG1 monoclonal antibody directed to N3pGlu Aβ epitope and targets removal of existing amyloid plaque. The methods of making and using LY3372993 are described in U.S. Pat. Nos. 10,647,759 and 11,078,261 (referred to as antibody 201c therein), which are expressly incorporated herein by reference in their entirety. In some embodiments, the antibody has the following sequence.

Light Chain:
SEQ ID NO. 3
DIQMTQSPSTLSASVGDRVTITCRASQSLGNWLAWYQQKPGKAPKLLIY
QASTLESGVPSRFSGSGSGTEFTLTISSLQPDDFATYYCQHYKGSFWTF
GQGTKVEIKRTVAAPSVFIFPPSDEQLKSGTASVVCLLNNFYPREAKVQ
WKVDNALQSGNSQESVTEQDSKDSTYSLSSTLTLSKADYEKHKVYACEV
THQGLSSPVTKSFNRGEC
Heavy Chain:
SEQ ID NO. 4
EVQLLESGGGLVQPGGSLRLSCAASGFTFSSYPMSWVRQAPGKGLEWVS
AISGSGGSTYYADSVKGRFTISRDNSKNTLYLQMNSLRAEDTAVYYCAR
EGGSGSYYNGFDYWGQGTLVTVSSASTKGPSVFPLAPSSKSTSGGTAAL
GCLVKDYFPEPVTVSWNSGALTSGVHTFPAVLQSSGLYSLSSVVTVPSS
SLGTQTYICNVNHKPSNTKVDKKVEPKSCDKTHTCPPCPAPELLGGPSV
FLFPPKPKDTLMISRTPEVTCVVVDVSHEDPEVKFNWYVDGVEVHNAKT
KPREEQYNSTYRVVSVLTVLHQDWLNGKEYKCKVSNKALPAPIEKTISK
AKGQPREPQVYTLPPSRDELTKNQVSLTCLVKGFYPSDIAVEWESNGQP
ENNYKTTPPVLDSDGSFFLYSKLTVDKSRWQQGNVFSCSVMHEALHNHY
TQKSLSLSPG

ADUHELM® (aducanumab-avwa) is an amyloid beta-directed antibody indicated to treat Alzheimer's disease. Suitable dosage includes 10 mg/kg administered as an intravenous infusion every four weeks.

Solanezumab is a monoclonal antibody that selectively binds to soluble amyloid-beta (Aβ) to promote Aβ clearance. Suitable dosages of solanezumab include 400 mg to 1600 mg (preferably, 1600 mg) intravenously every four weeks. Solanezumab is described in (including methods of making and using it) in the following patent documents, which are expressly incorporated herein by reference: U.S. Pat. No. 7,195,761, US Patent Application Publication No. 20060039906, U.S. Pat. Nos. 7,892,545, 8,591,894,7,771,722, US Patent Application Publication No. 20070190046. Skilled artisans will recognize that solanezumab is an IgG1 monoclonal antibody having CDRs. Solanezumab binds to the mid-domain of the Aβ peptide. In some embodiments, the antibody has the following sequence.

Light Chain:
SEQ. ID NO. 5
DVVMTQSPLSLPVTLGQPASISCRSSQSLIYSDGNAYLHWFLQKPGQSPR
LLIYKVSNRFSGVPDRFSGSGSGTDFTLKISRVEAEDVGVYYCSQSTHVP
WTFGQGTKVEIKRTVAAPSVFIFPPSDEQLKSGTASVVCLLNNFYPREAK
VQWKVDNALQSGNSQESVTEQDSKDSTYSLSSTLTLSKADYEKHKVYACE
VTHQGLSSPVTKSFNRGEC
Heavy Chain:
SEQ. ID NO. 6
EVQLVESGGGLVQPGGSLRLSCAASGFTFSRYSMSWVRQAPGKGLELVAQ
INSVGNSTYYPDTVKGRFTISRDNAKNTLYLQMNSLRAEDTAVYYCASGD
YWGQGTLVTVSSASTKGPSVFPLAPSSKSTSGGTAALGCLVKDYFPEPVT
VSWNSGALTSGVHTFPAVLQSSGLYSLSSVVTVPSSSLGTQTYICNVNHK
PSNTKVDKKVEPKSCDKTHTCPPCPAPELLGGPSVFLFPPKPKDTLMISR
TPEVTCVVVDVSHEDPEVKFNWYVDGVEVHNAKTKPREEQYNSTYRVVSV
LTVLHQDWLNGKEYKCKVSNKALPAPIEKTISKAKGQPREPQVYTLPPSR
DELTKNQVSLTCLVKGFYPSDIAVEWESNGQPENNYKTTPPVLDSDGSFF
LYSKLTVDKSRWQQGNVFSCSVMHEALHNHYTQKSLSLSPGK

The antibody of the preceding sequence (as well the antibodies of the patents noted above in the following patent documents U.S. Pat. No. 7,195,761, US Patent Application Publication No. 20060039906, U.S. Pat. Nos. 7,892,545,8,591,894, 7,771,722, US Patent Application Publication No. 20070190046) may be formulated and referred to as solanezumab. Thus, the present embodiments include antibodies with SEQ ID. 1 and 2 or other antibodies, as well as those antibodies which have been formulated in a composition called solanezumab. Those skilled in the art will appreciate that either may be used.

Lecanemab (BAN2401) is a humanized monoclonal antibody administered to slow down the progression of Alzheimer's disease by neutralizing and eliminating soluble, Aβ aggregates (protofibrils) that may contribute to the neurodegenerative process. Suitable dosage of lecanemab includes 10 mg/kg of body weight.

LY3372689, N-[4-fluoro-5-[[(2S,4S)-2-methyl-4-[(5-methyl-1,2,4- oxadiazol-3-yl)-methoxy]-1-piperidyl]methyl]thiazol-2-yl]acetamide, is a small-molecule inhibitor of O-GlcNAcase, which is believed to reduce tau pathology and associated neurodegeneration. Methods of preparing and using LY3372689 are described in U.S. Pat. No. 10,081,625, which is expressly incorporated herein by reference. The skilled artisan will recognize that pharmaceutically acceptable salts of LY3372689 may be prepared for pharmaceutical use, and formulations with one or more pharmaceutically acceptable carriers, diluents, or excipients may be used in the manufacture of a medicament. Thus, the present embodiments include the structure of LY3372689 below, which may be prepared as a pharmaceutically acceptable salt form and formulated in a composition called LY3372689. Those skilled in the art will appreciate that any may be used. Dose of this molecule is disclosed in WO 2022/020663.

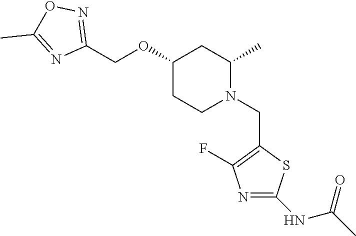 [SMILES: CC(=O)Nc1nc(F)c(CN2CC[C@H](OCc3noc(C)n3)C[C@@H]2C)s1]

Suitable brain regions are described herein.

When the brain region is inferior temporal brain region, the tau-PET SUVR ranges from about 1.05 to about 1.45. In some embodiments, when the brain region is inferior temporal brain region, the tau-PET SUVR is about 1.45. When the brain region is lateral temporal brain region, the tau-PET SUVR ranges from about 1.35 to about 1.45. When the brain region is middle and superior temporal brain region, the tau-PET SUVR ranges from about 1.35 to about 1.45. When the brain region is lateral parietal brain region, the tau-PET SUVR ranges from about 1.10 to about 1.45. When the brain region is bilateral entorhinal cortex, the tau-PET SUVR ranges from about 1.05 to about 1.45. When the brain region is fusiform, the tau-PET SUVR ranges from about 1.05 to about 1.45. When the brain region is parahippocampal, the tau-PET SUVR ranges from about 1.05 to about 1.45.

In some embodiments, the method can further include analyzing an amyloid-positron emission tomography (PET) scan to determine amyloid status.

In some embodiments, the method can further include determining cortical thickness.

In some embodiments, the method can further include analyzing cerebrospinal fluid for amyloid-β.

In some embodiments, the method can further include analyzing epsilon-4 allele of apolipoprotein E (APOE E4) genotype.

In some embodiments, the method can further include obtaining a tau-positron emission tomography (PET) scan from a brain region of the patient and analyzing the tau-PET scan to determine a tau-PET SUVR following administration of the Alzheimer's disease therapy.

In another aspect, the present disclosure is directed to a method of diagnosing a patient as having or suspected of having Alzheimer's disease. The method includes: analyzing a tau-positron emission tomography (PET) scan from a brain region of the patient to determine a tau-PET SUVR; and diagnosing the patient as having or as suspected of having Alzheimer's disease if the tau-PET SUVR ranges from about 1.10 to about 1.45.

Suitable brain regions are described herein.

In some embodiments, the patient is diagnosed as having or as suspected of having Alzheimer's disease if the brain region is inferior temporal brain region and the tau-PET SUVR is 1.45. In some embodiments, the patient is diagnosed as having or as suspected of having Alzheimer's disease if the brain region is lateral temporal brain region and the tau-PET SUVR ranges from about 1.35 to about 1.45. In some embodiments, the patient is diagnosed as having or as suspected of having Alzheimer's disease if the brain region is middle and superior temporal brain region and the tau-PET SUVR ranges from about 1.35 to about 1.45. In some embodiments, the patient is diagnosed as having or as suspected of having Alzheimer's disease if the brain region is lateral parietal brain region and the tau-PET SUVR ranges from about 1.10 to about 1.45.

The method can further include analyzing an amyloid-positron emission tomography (PET) scan to determine amyloid status.

In some embodiments, the method can further include determining cortical thickness.

In some embodiments, the method can further include analyzing cerebrospinal fluid for amyloid-β.

In some embodiments, the method can further include analyzing epsilon-4 allele of apolipoprotein E (APOE E4) genotype.

In some embodiments, the patient has or is suspected of having amyloid plaques.

In some embodiments, the patient has or is suspected of having Alzheimer's disease cognitive decline.

In some embodiments, the method can further include administering an Alzheimer's disease therapy and disclosed herein.

In some embodiments, the method further includes obtaining a tau-positron emission tomography (PET) scan from a brain region of the patient and analyzing the tau-PET scan to determine a tau-PET SUVR following administration of the Alzheimer's disease therapy.

In another aspect, the present disclosure is directed to a method of identifying a patient having or suspected of having Alzheimer's disease as a candidate patient for receiving an Alzheimer's disease therapy, the method comprising: analyzing a tau-positron emission tomography (PET) scan from a brain region of the patient to determine a tau-PET SUVR; and identifying the patient as a candidate patient for receiving an Alzheimer's disease therapy if the tau-PET SUVR ranges from about 1.10 to about 1.45.

Suitable brain regions are described herein.

In some embodiments, the patient is identified as a candidate patient for receiving Alzheimer's disease therapy if the brain region is inferior temporal brain region and the tau-PET SUVR is 1.45. In some embodiments, the patient is identified as a candidate patient for receiving Alzheimer's disease therapy if the brain region is lateral temporal brain region and the tau-PET SUVR ranges from about 1.35 to about 1.45. In some embodiments, the patient is identified as a candidate patient for receiving Alzheimer's disease therapy if the brain region is middle and superior temporal brain region and the tau-PET SUVR ranges from about 1.35 to about 1.45. In some embodiments, the patient is identified as a candidate patient for receiving Alzheimer's disease therapy if the brain region is lateral parietal brain region and the tau-PET SUVR ranges from about 1.10 to about 1.45.

The method can further include analyzing an amyloid-positron emission tomography (PET) scan to determine amyloid status.

In some embodiments, the method can further include determining cortical thickness.

In some embodiments, the method can further include analyzing cerebrospinal fluid for amyloid-β.

In some embodiments, the method can further include analyzing epsilon-4 allele of apolipoprotein E (APOE E4) genotype.

In some embodiments, the patient has or is suspected of having amyloid plaques.

In some embodiments, the patient has or is suspected of having Alzheimer's disease cognitive decline.

In some embodiments, the method can further include administering an Alzheimer's disease therapy and disclosed herein.

In some embodiments, the method further includes obtaining a tau-positron emission tomography (PET) scan from a brain region of the patient and analyzing the tau-PET scan to determine a tau-PET SUVR following administration of the Alzheimer's disease therapy.

In another aspect, the present disclosure is directed to a method of treating a patient having or suspected of having amyloid plaques. The method comprising: analyzing a tau image of a brain region of the patient to determine a tau-PET SUVR; and administering an Alzheimer's disease therapy to the patient if the tau-PET SUVR ranges from about 1.10 to about 1.45.

Suitable brain regions are described herein.

In some embodiments, the patient is identified as having amyloid plaques if the brain region is inferior temporal brain region and the tau-PET SUVR is 1.45. In some embodiments, the patient is identified as having amyloid plaques if the brain region is lateral temporal brain region and the tau-PET SUVR ranges from about 1.35 to about 1.45. In some embodiments, the patient is identified as having amyloid plaques if the brain region is middle and superior temporal brain region and the tau-PET SUVR ranges from about 1.35 to about 1.45. In some embodiments, the patient is identified as having amyloid plaques if the brain region is lateral parietal brain region and the tau-PET SUVR ranges from about 1.10 to about 1.45.

The method can further include analyzing an amyloid-positron emission tomography (PET) scan to determine amyloid status.

In some embodiments, the method can further include determining cortical thickness.

In some embodiments, the method can further include analyzing cerebrospinal fluid for amyloid-β.

In some embodiments, the method can further include analyzing epsilon-4 allele of apolipoprotein E (APOE E4) genotype.

In some embodiments, the patient has or is suspected of having Alzheimer's disease cognitive decline.

In some embodiments, the method can further include administering an Alzheimer's disease therapy as disclosed herein.

In some embodiments, the method further includes obtaining a tau-positron emission tomography (PET) scan from a brain region of the patient and analyzing the tau-PET scan to determine a tau-PET SUVR following administration of the Alzheimer's disease therapy.

In one aspect, the present disclosure is directed to a method of diagnosing a patient as having or suspected of having amyloid plaques, the method comprising: analyzing a tau-positron emission tomography (PET) scan from a brain region of the patient to determine a tau-PET SUVR; and diagnosing the patient as having or as suspected of having amyloid plaques if the tau-PET SUVR ranges from about 1.10 to about 1.45.

Suitable brain regions are described herein.

In some embodiments, the patient is identified as having amyloid plaques if the brain region is inferior temporal brain region and the tau-PET SUVR is 1.45. In some embodiments, the patient is identified as having amyloid plaques if the brain region is lateral temporal brain region and the tau-PET SUVR ranges from about 1.35 to about 1.45. In some embodiments, the patient is identified as having amyloid plaques if the brain region is middle and superior temporal brain region and the tau-PET SUVR ranges from about 1.35 to about 1.45. In some embodiments, the patient is identified as having amyloid plaques if the brain region is lateral parietal brain region and the tau-PET SUVR ranges from about 1.10 to about 1.45.

The method can further include analyzing an amyloid-positron emission tomography (PET) scan to determine amyloid status.

In some embodiments, the method can further include determining cortical thickness.

In some embodiments, the method can further include analyzing cerebrospinal fluid for amyloid-β.

In some embodiments, the method can further include analyzing epsilon-4 allele of apolipoprotein E (APOE E4) genotype.

In some embodiments, the patient has or is suspected of having Alzheimer's disease cognitive decline.

In some embodiments, the method can further include administering an Alzheimer's disease therapy as disclosed herein.

In some embodiments, the method further includes obtaining a tau-positron emission tomography (PET) scan from a brain region of the patient and analyzing the tau-PET scan to determine a tau-PET SUVR following administration of the Alzheimer's disease therapy.

In one aspect, the present disclosure is directed to a method of identifying a patient having or suspected of having amyloid plaques as a candidate patient for receiving an Alzheimer's disease therapy, the method comprising: analyzing a tau-positron emission tomography (PET) scan from a brain region of the patient to determine a tau-PET SUVR; and identifying the patient as a candidate patient for receiving an Alzheimer's disease therapy if the tau-PET SUVR ranges from about 1.10 to about 1.45.

Suitable brain regions are described herein.

In some embodiments, the patient is identified as having amyloid plaques if the brain region is inferior temporal brain region and the tau-PET SUVR is 1.45. In some embodiments, the patient is identified as having amyloid plaques if the brain region is lateral temporal brain region and the tau-PET SUVR ranges from about 1.35 to about 1.45. In some embodiments, the patient is identified as having amyloid plaques if the brain region is middle and superior temporal brain region and the tau-PET SUVR ranges from about 1.35 to about 1.45. In some embodiments, the patient is identified as having amyloid plaques if the brain region is lateral parietal brain region and the tau-PET SUVR ranges from about 1.10 to about 1.45.

The method can further include analyzing an amyloid-positron emission tomography (PET) scan to determine amyloid status.

In some embodiments, the method can further include determining cortical thickness.

In some embodiments, the method can further include analyzing cerebrospinal fluid for amyloid-β.

In some embodiments, the method can further include analyzing epsilon-4 allele of apolipoprotein E (APOE E4) genotype.

In some embodiments, the patient has or is suspected of having Alzheimer's disease cognitive decline.

In some embodiments, the method can further include administering an Alzheimer's disease therapy as disclosed herein.

In some embodiments, the method further includes obtaining a tau-positron emission tomography (PET) scan from a brain region of the patient and analyzing the tau-PET scan to determine a tau-PET SUVR following administration of the Alzheimer's disease therapy.

In another aspect, the present disclosure is directed to a method of treating a patient having or suspected of having Alzheimer's disease cognitive decline. The method includes: analyzing a tau image of a brain region of the patient to determine a tau-PET SUVR; and administering an Alzheimer's disease therapy to the patient if the tau-PET SUVR ranges from about 1.10 to about 1.45.

Suitable brain regions are described herein.

In some embodiments, the patient is identified as having Alzheimer's disease cognitive decline if the brain region is inferior temporal brain region and the tau-PET SUVR is 1.45. In some embodiments, the patient is identified as having Alzheimer's disease cognitive decline if the brain region is lateral temporal brain region and the tau-PET SUVR ranges from about 1.35 to about 1.45. In some embodiments, the patient is identified as having Alzheimer's disease cognitive decline if the brain region is middle and superior temporal brain region and the tau-PET SUVR ranges from about 1.35 to about 1.45. In some embodiments, the patient is identified as having Alzheimer's disease cognitive decline if the brain region is lateral parietal brain region and the tau-PET SUVR ranges from about 1.10 to about 1.45.

The method can further include analyzing an amyloid-positron emission tomography (PET) scan to determine amyloid status.

In some embodiments, the method can further include determining cortical thickness.

In some embodiments, the method can further include analyzing cerebrospinal fluid for amyloid-β.

In some embodiments, the method can further include analyzing epsilon-4 allele of apolipoprotein E (APOE E4) genotype.

In some embodiments, the patient has or is suspected of having amyloid plaques.

In some embodiments, the method can further include administering an Alzheimer's disease therapy as disclosed herein.

In some embodiments, the method further includes obtaining a tau-positron emission tomography (PET) scan from a brain region of the patient and analyzing the tau-PET scan to determine a tau-PET SUVR following administration of the Alzheimer's disease therapy.

In one aspect, the present disclosure is directed to a method of diagnosing a patient as having or suspected of having Alzheimer's disease cognitive decline. The method includes: analyzing a tau-positron emission tomography (PET) scan from a brain region of the patient to determine a tau-PET SUVR; and diagnosing the patient as having or as suspected of having Alzheimer's disease cognitive decline if the tau-PET SUVR ranges from about 1.10 to about 1.45.

Suitable brain regions are described herein.

In some embodiments, the patient is identified as having Alzheimer's disease cognitive decline if the brain region is inferior temporal brain region and the tau-PET SUVR is 1.45. In some embodiments, the patient is identified as having Alzheimer's disease cognitive decline if the brain region is lateral temporal brain region and the tau-PET SUVR ranges from about 1.35 to about 1.45. In some embodiments, the patient is identified as having Alzheimer's disease cognitive decline if the brain region is middle and superior temporal brain region and the tau-PET SUVR ranges from about 1.35 to about 1.45. In some embodiments, the patient is identified as having Alzheimer's disease cognitive decline if the brain region is lateral parietal brain region and the tau-PET SUVR ranges from about 1.10 to about 1.45.

The method can further include analyzing an amyloid-positron emission tomography (PET) scan to determine amyloid status.

In some embodiments, the method can further include determining cortical thickness.

In some embodiments, the method can further include analyzing cerebrospinal fluid for amyloid-β.

In some embodiments, the method can further include analyzing epsilon-4 allele of apolipoprotein E (APOE E4) genotype.

In some embodiments, the patient has or is suspected of having amyloid plaques.

In some embodiments, the method can further include administering an Alzheimer's disease therapy as disclosed herein.

In some embodiments, the method further includes obtaining a tau-positron emission tomography (PET) scan from a brain region of the patient and analyzing the tau-PET scan to determine a tau-PET SUVR following administration of the Alzheimer's disease therapy.

In one aspect, the present disclosure is directed to a method of identifying cognitive decline in a patient having or suspected of having Alzheimer's disease, the method comprising: analyzing a tau-positron emission tomography (PET) scan from a brain region of the patient to determine a tau-PET SUVR; and identifying the patient as having cognitive decline if the tau-PET SUVR ranges from about 1.10 to about 1.45.

In some embodiments, the patient is identified as having cognitive decline if the brain region is inferior temporal brain region and the tau-PET SUVR is 1.45. In some embodiments, the patient is identified as having cognitive decline if the brain region is lateral temporal brain region and the tau-PET SUVR ranges from about 1.35 to about 1.45. In some embodiments, the patient is identified as having cognitive decline if the brain region is middle and superior temporal brain region and the tau-PET SUVR ranges from about 1.35 to about 1.45. In some embodiments, the patient is identified as having cognitive decline if the brain region is lateral parietal brain region and the tau-PET SUVR ranges from about 1.10 to about 1.45.

The method can further include analyzing an amyloid-positron emission tomography (PET) scan to determine amyloid status.

In some embodiments, the method can further include determining cortical thickness.

In some embodiments, the method can further include analyzing cerebrospinal fluid for amyloid-β.

In some embodiments, the method can further include analyzing epsilon-4 allele of apolipoprotein E (APOE E4) genotype.

In some embodiments, the patient has or is suspected of having amyloid plaques.

In some embodiments, the method can further include administering an Alzheimer's disease therapy as disclosed herein.

In some embodiments, the method further includes obtaining a tau-positron emission tomography (PET) scan from a brain region of the patient and analyzing the tau-PET scan to determine a tau-PET SUVR following administration of the Alzheimer's disease therapy.

In another aspect, the present disclosure is directed to a method of identifying whether a patient receiving an Alzheimer's disease therapy is responding to the Alzheimer's disease therapy. The method includes: analyzing a tau-positron emission tomography (PET) scan from a brain region of the patient to determine a tau-PET SUVR; administering an Alzheimer's disease therapy to the patient; analyzing a tau-positron emission tomography (PET) scan from the brain region of the patient to determine a tau-PET SUVR after administering the Alzheimer's disease therapy; and identifying the patient as responding to the Alzheimer's disease therapy if the tau-PET SUVR before administering the Alzheimer's disease therapy is changed based on determination of the tau-PET SUVR after administering the Alzheimer's disease therapy.

Alzheimer's disease therapies are described herein.

Suitable brain regions are described herein.

In some embodiments, the patient is identified as responding if the brain region is inferior temporal brain region and the tau-PET SUVR is 1.45. In some embodiments, the patient is identified as responding if the brain region is lateral temporal brain region and the tau-PET SUVR ranges from about 1.35 to about 1.45. In some embodiments, the patient is identified as responding if the brain region is middle and superior temporal brain region and the tau-PET SUVR ranges from about 1.35 to about 1.45. In some embodiments, the patient is identified as responding if the brain region is lateral parietal brain region and the tau-PET SUVR ranges from about 1.10 to about 1.45.

The method can further include analyzing an amyloid-positron emission tomography (PET) scan to determine amyloid status.

In some embodiments, the method can further include determining cortical thickness.

In some embodiments, the method can further include analyzing cerebrospinal fluid for amyloid-β.

In some embodiments, the method can further include analyzing epsilon-4 allele of apolipoprotein E (APOE E4) genotype.

The method can further include analyzing the patient for amyloid plaques before and after administering the Alzheimer's disease therapy.

The method can further include analyzing the patient for cognitive decline before and after administering the Alzheimer's disease therapy.

In another aspect, the present disclosure is directed to a method of determining tau burden in a patient having or suspected of having Alzheimer's disease. The method includes: analyzing a tau-positron emission tomography (PET) scan from a brain region of the patient to determine a tau-PET SUVR; and identifying the patient as having tau burden if the tau-PET SUVR is greater than 1.15.

Suitable brain regions are described herein.

The method can further include analyzing an amyloid-positron emission tomography (PET) scan to determine amyloid status.

In some embodiments, the method can further include determining cortical thickness.

In some embodiments, the method can further include analyzing cerebrospinal fluid for amyloid-β.

In some embodiments, the method can further include analyzing epsilon-4 allele of apolipoprotein E (APOE E4) genotype.

In some embodiments, the patient has or is suspected of having amyloid plaques.

In some embodiments, the method can further include administering an Alzheimer's disease therapy as disclosed herein.

In some embodiments, the method further includes obtaining a tau-positron emission tomography (PET) scan from a brain region of the patient and analyzing the tau-PET scan to determine a tau-PET SUVR following administration of the Alzheimer's disease therapy.

In one embodiment, the brain region is inferior temporal brain region and the tau-PET SUVR is greater than 1.40. In one embodiment, the brain region is lateral temporal brain region and the tau-PET SUVR is greater than 1.30. In one embodiment, the brain region is middle and superior temporal brain region and the tau-PET SUVR is greater than 1.30. In one embodiment, the brain region is lateral parietal brain region and the tau-PET SUVR is greater than 1.15.

In one aspect, the present disclosure is directed to a method for determining whether a patient is a candidate for enrollment in an Alzheimer's disease clinical trial. The method includes: analyzing a tau-positron emission tomography (PET) scan from a brain region of the patient to determine a region-based tau-PET SUVR; and identifying the patient as a candidate for enrollment if the tau-PET SUVR is greater than 1.15.

Suitable brain regions are described herein.

In some embodiments, the patient is identified as a candidate for enrollment if the brain region is inferior temporal brain region and the tau-PET SUVR is 1.45. In some embodiments, the patient is identified a candidate for enrollment if the brain region is lateral temporal brain region and the tau-PET SUVR ranges from about 1.35 to about 1.45. In some embodiments, the patient is identified as a candidate for enrollment if the brain region is middle and superior temporal brain region and the tau-PET SUVR ranges from about 1.35 to about 1.45. In some embodiments, the patient is identified as a candidate for enrollment if the brain region is lateral parietal brain region and the tau-PET SUVR ranges from about 1.10 to about 1.45.

The method can further include analyzing an amyloid-positron emission tomography (PET) scan to determine amyloid status.

In some embodiments, the method can further include determining cortical thickness.

In some embodiments, the method can further include analyzing cerebrospinal fluid for amyloid-β.

In some embodiments, the method can further include analyzing epsilon-4 allele of apolipoprotein E (APOE E4) genotype.

In some embodiments, the patient has or is suspected of having amyloid plaques.

In some embodiments, the method can further include administering an Alzheimer's disease therapy as disclosed herein.

In some embodiments, the method further includes obtaining a tau-positron emission tomography (PET) scan from a brain region of the patient and analyzing the tau-PET scan to determine a tau-PET SUVR following administration of the Alzheimer's disease therapy.

In some embodiments, a patient is identified as a candidate patient for receiving an Alzheimer's disease therapy if the tau-PET SUVR ranges from about 1.10 to about 1.45. In some embodiments, a patient is identified as a candidate patient for receiving an Alzheimer's disease therapy if the tau-PET SUVR is about 1.10. In some embodiments, a patient is identified as a candidate patient for receiving an Alzheimer's disease therapy if the tau-PET SUVR is about 1.15. In some embodiments, a patient is identified as a candidate patient for receiving an Alzheimer's disease therapy if the tau-PET SUVR is about 1.20. In some embodiments, a patient is identified as a candidate patient for receiving an Alzheimer's disease therapy if the tau-PET SUVR is about 1.25. In some embodiments, a patient is identified as a candidate patient for receiving an Alzheimer's disease therapy if the tau-PET SUVR is about 1.30. In some embodiments, a patient is identified as a candidate patient for receiving an Alzheimer's disease therapy if the tau-PET SUVR is about 1.35. In some embodiments, a patient is identified as a candidate patient for receiving an Alzheimer's disease therapy if the tau-PET SUVR is about 1.40. In some embodiments, a patient is identified as a candidate patient for receiving an Alzheimer's disease therapy if the tau-PET SUVR is about 1.45.

### EXAMPLES

### Example 1. Prognostic Value of Regional Tau PET Measures in Alzheimer's Disease

Datasets:

### A05 Trial Dataset (NCT02016560)

N=35 AD and N=73 participants
Subjects in the Confirmatory Cohort (A05C) of ¹⁸F-AV-1451-A05 (flortaucipir) phase 2/3 study
Subjects that had 18 months follow-up data for 11-item Alzheimer's disease Assessment Scale-Cognitive subscale (ADAS-Cog11) score

### ADNI Dataset

N=24 AD and N=76 MCI participants
Subjects that had a baseline ADAS-Cog13 scoring along with 2 to 3 years of longitudinal follow-up
Subjects who had at least one tau PET (flortaucipir) scan at baseline and a florbetapir (FBP) scan within 6 months of baseline ADAS-Cog13 scoring
ADNI and A05 cohort demographics are shown in the table below.

| | Cohort | | | |
|---|---|---|---|---|
| | ADNI (N = 100) | | A05 (N = 108) | |
| | AD | MCI | AD | MCI |
| Demographic | (N = 24) | (N = 76) | (N = 35) | (N = 73) |
| Age, years, | 76.9 (8.4) | 77.1 (7.3) | 73.8 (9.9) | 73.4 (9.7) |
| mean (SD) | | | | |
| Baseline | 21.8 (3.5) | 27.8 (2.2) | 23.6 (2.4) | 25.8 (1.5) |
| MMSE, mean | | | | |
| (SD) | | | | |
| ADAS-cog*, | 4.9 (4.8) | 0.8 (2.0) | 4.8 (5.4) | 1.7 (4.3) |
| annual change, | | | | |
| mean (SD) | | | | |
| MUBADA | 1.53 (0.6) | 1.24 (0.2) | 1.48 (0.5) | 1.30 (0.4) |
| SUVR (ref: | | | | |
| cereCrus), | | | | |
| mean (SD) | | | | |
| MUBADA | n/a | n/a | 1.27 (0.3) | 1.12 (0.2) |
| SUVR, (ref: | | | | |
| PERSI), mean | | | | |
| (SD) | | | | |
| Amyloid | 22 (92) | 38 (50) | 28 (80) | 43 (59) |
| positive by | | | | |
| FBP, N (%) | | | | |

Method:

Tau burden (standardized uptake value ratio, SUVR) was quantified using AAL brain atlas-based temporal and parietal regions and AD-specific weighted cortical composite region (MUBADA) with reference to cerebellar crus region (cereCrus) and Parametric Estimation of Reference Signal Intensity in white matter (PERSI).

For every global and region-based tau SUVR, subjects were dichotomized into tau groups of high (T+) and low (T−) by using a series of thresholds (1.0-1.5). Signal-to-noise ratios (SNRs) of the cognitive decline and florbetapir-based amyloid positivity (quantitative threshold) were assessed for every T+ group based on regional and composite tau PET measures. SNR was defined as: Mean annual ADAS-Cog Score change/ADAS-Cog Score standard deviation.

Results:

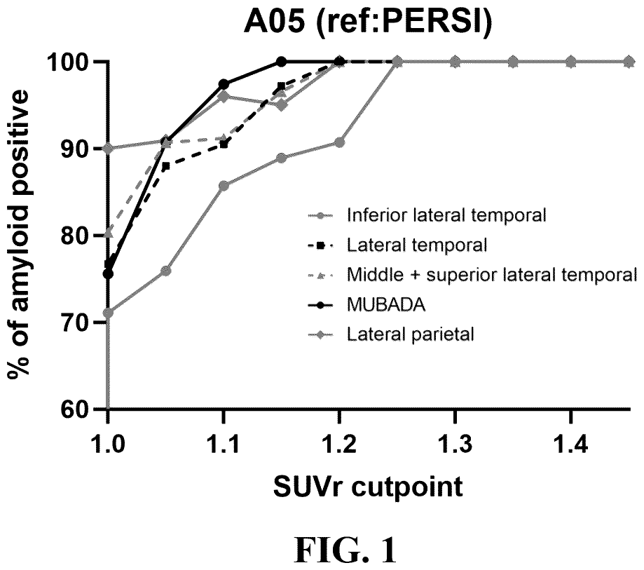

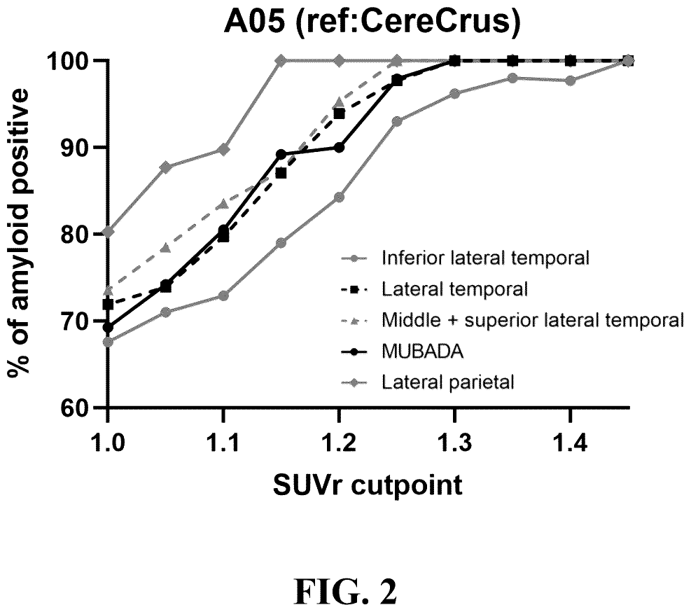

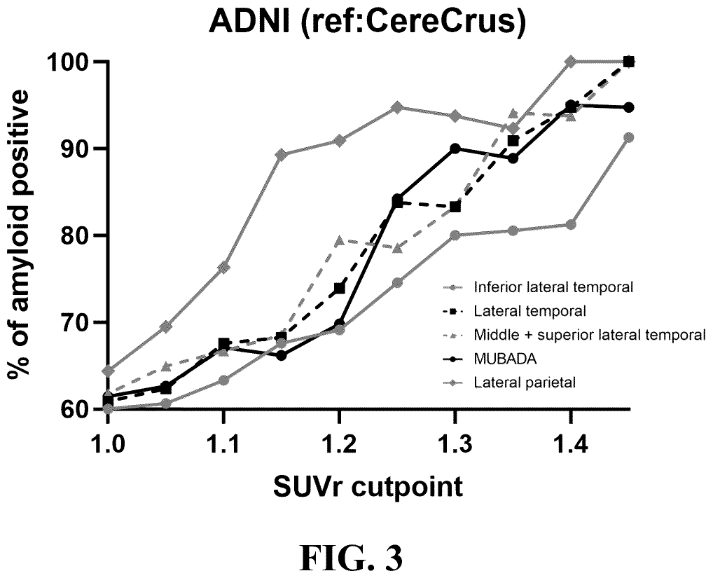

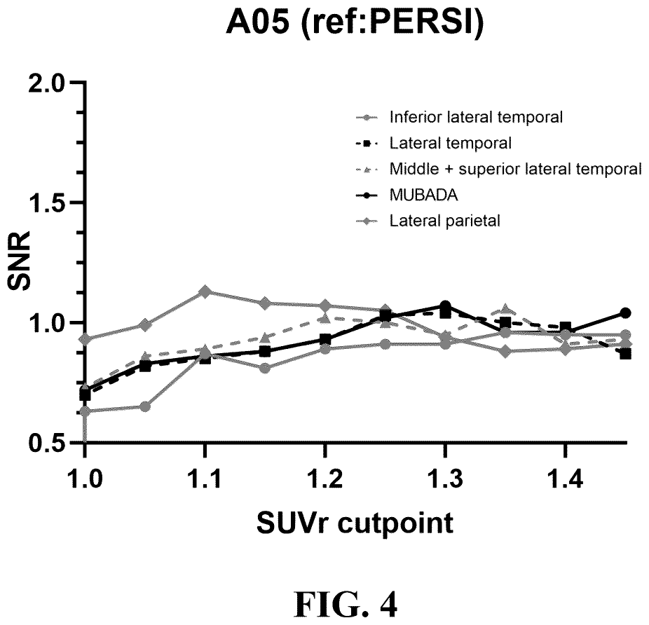

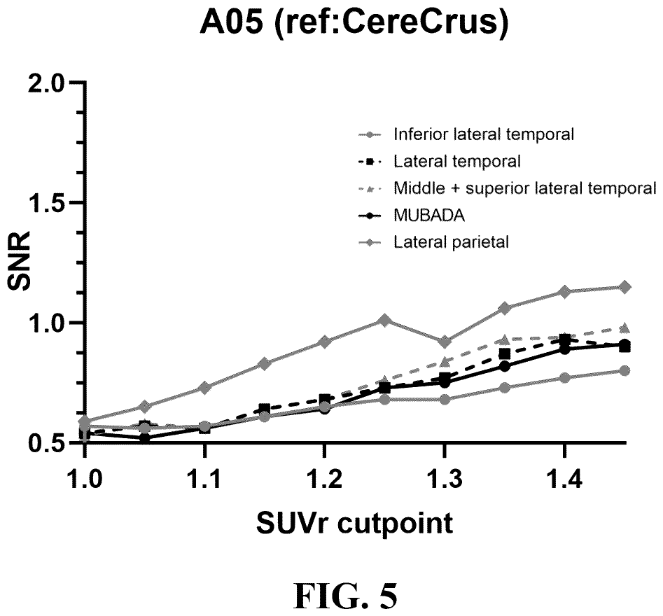

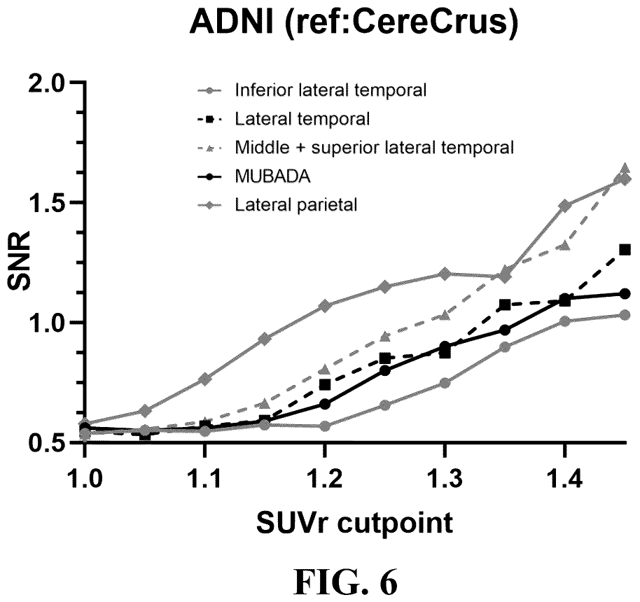

As represented by the data in FIGS. 1-6, regional tau PET measures are prognostic of cognitive decline and predictive of florbetapir (FBP) assessed amyloid positivity.

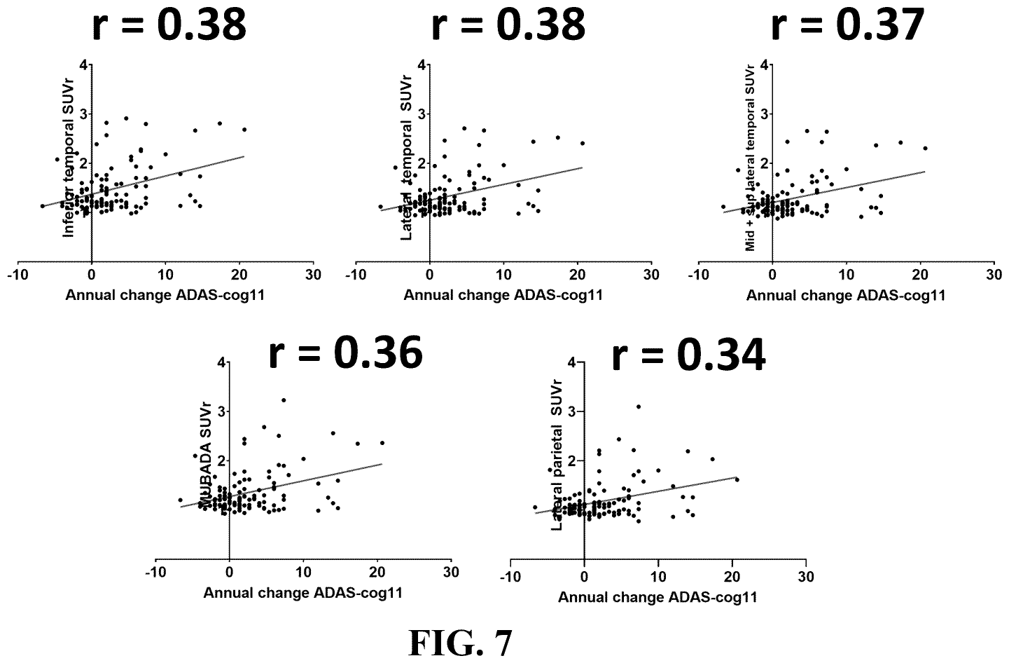

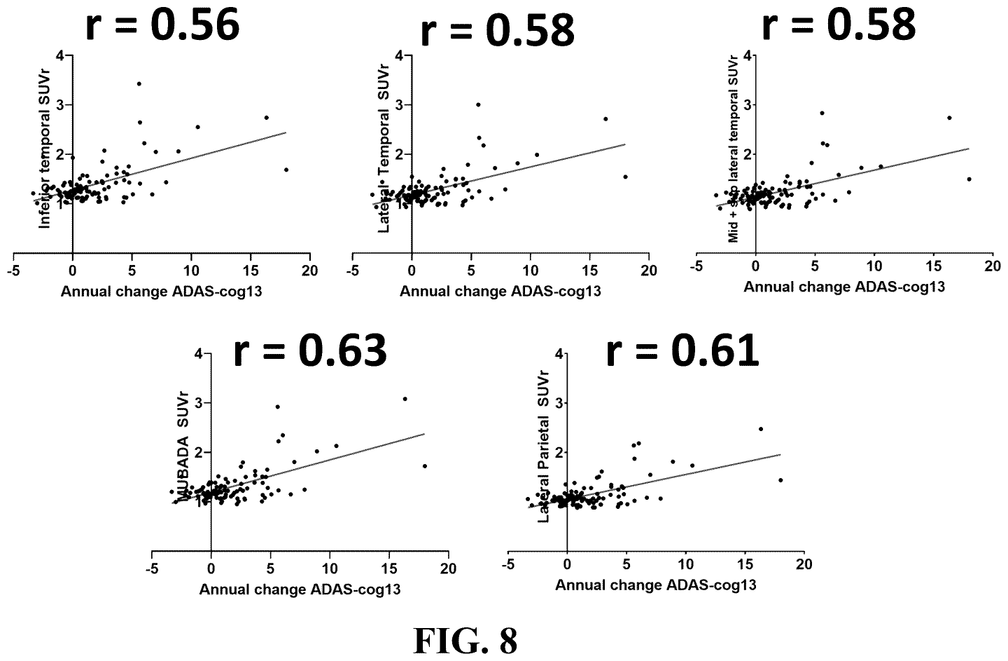

Regional tau PET analysis showed that utilizing temporal and parietal SUVR values could help stratify patients by risk of cognitive decline and amyloid status and, therefore, clinical trial eligibility algorithms can be implemented using regional tau metrics. Regional tau PET measures correlate with annual cognitive change. (Data from the A05 dataset is represented in FIG. 7 and data from the ADNI dataset is represented in FIG. 8, wherein “r” represents Pearson's correlation coefficient.) and may outperform composite tau PET measures in predicting cognitive decline and amyloid positivity.

Regional tau PET measures were highly correlated (>0.95 Pearson's r) with composite region tau PET measures.

For every global and region-based flortaucipir measure, an increase in SUVR threshold resulted in lower average MMSE score, larger average global tau burden, higher percentage of amyloid-positive patients and increased mean cognitive decline for patients in T+ group. SUVR thresholds that identified subgroups with high percentage (≥90%) of amyloid-positive (Table 1, below) and high (>1) SNRs (Table 2, below), were observed to be lower for brain regions identified later in the tau pathologic cascade: inferior temporal (1.45), lateral temporal (1.35), lateral parietal (1.20).

TABLE 1
N and percent of amyloid-positive participates in the high tau (T+) subgroup.

| | Cut point | | | | | | |
|---|---|---|---|---|---|---|---|
| Region | 1.15 | 1.20 | 1.25 | 1.30 | 1.35 | 1.40 | 1.45 |
| Inferior temporal, N (%) | 50 | 47 | 41 | 36 | 29 | 26 | 21 |
| | (67.6) | (69.1) | (74.6) | (80.0) | (80.6) | (81.3) | (91.3) |
| Lateral temporal, N (%) | 43 | 34 | 31 | 25 | 20 | 19 | 16 |
| | (68.3) | (73.9) | (83.8) | (83.3) | (90.9) | (94.7) | (100) |
| Middle + superior temporal, | 37 | 31 | 22 | 20 | 16 | 15 | 11 |
| N (%) | (68.5) | (79.5) | (78.6) | (83.3) | (94.1) | (93.8) | (100) |
| MUBADA, N (%) | 45 | 37 | 32 | 27 | 24 | 19 | 18 |
| | (66.2) | (69.8) | (84.2) | (90.0) | (88.9) | (95.0) | (94.7) |
| Lateral parietal, N (%) | 25 | 20 | 18 | 15 | 12 | 11 | 10 |
| | (89.3) | (90.9) | (94.7) | (93.8) | (92.3) | (100) | (100) |

TABLE 2
SNRs for change in ADAS-Cog₁₃ in high tau (T+) subgroup.

| | Cut point | | | | | | |
|---|---|---|---|---|---|---|---|
| Region | 1.15 | 1.20 | 1.25 | 1.30 | 1.35 | 1.40 | 1.45 |
| Inferior temporal, SNR | 0.57 | 0.57 | 0.66 | 0.75 | 0.90 | 1.01 | 1.03 |
| Lateral temporal, SNR | 0.59 | 0.74 | 0.85 | 0.87 | 1.07 | 1.09 | 1.30 |
| Middle + superior temporal, | 0.66 | 0.81 | 0.94 | 1.03 | 1.22 | 1.32 | 1.65 |
| SNR | | | | | | | |
| MUBADA, SNR | 0.59 | 0.66 | 0.80 | 0.90 | 0.97 | 1.10 | 1.12 |
| Lateral parietal, SNR | 0.93 | 1.07 | 1.15 | 1.20 | 1.19 | 1.49 | 1.60 |

The following references are incorporated herein by reference. [1] Ossenkoppele et al, JAMA Neurology, 2021 [2] Pontecorvo et al, Brain, 2019; [3] Devous et al, Journal of Nuclear medicine, 2018; [4] Southekal et al, Journal of Nuclear medicine, 2018.

### Example 2. Early Tau Detection in Flortaucipir Images-Validation in Autopsy-Confirmed Data and Implications for Disease Progression

There is an increasing interest in utilizing tau PET to identify patients early in Alzheimer's disease (AD). In this example, a temporal lobe composite (Eτ) VOI was evaluated in a longitudinal flortaucipir cohort and compared to a previously described global neocortical region. In a separate autopsy-confirmed study, the sensitivity of the Eτ VOI for identifying intermediate (B2) neurofibrillary tangle (NFT) pathology was evaluated.

Methods: 427 subjects received flortaucipir, florbetapir, MRI, and cognitive evaluation at baseline and 18-months.

TABLE 3
Demographics of subjects that were retrospectively analyzed.

| | YCN/Aβ− | CN/Aβ− | CN/Aβ+ | MCI/Aβ− | MCI/Aβ+ | AD/Aβ− | AD/Aβ+ |
|---|---|---|---|---|---|---|---|
| N | 16 | 53 | 5 | 50 | 47 | 16 | 240 |
| Age | 28.9 ± 4.9 | 67.6 ± 10.2 | 77.8 ± 7.0 | 69.1 ± 9.3 | 72.7 ± 9.1 | 72.2 ± 6.9 | 73.7 ± 8.3 |
| MMSE | 29.6 ± 0.5 | 29.5 ± 0.5 | 29.6 ± 0.5 | 28.2 ± 1.7 | 27.4 ± 1.8 | 22.9 ± 3.2 | 22.5 ± 2.9 |
| Sex | 7F/9M | 24F/29M | 2F/3M | 27F/23M | 21F/26M | 7F/9M | 135F/104M |
| Education | 15CD/1HS | 45CD/8HS | 4CD/1HS | 37CD/13HS | 38CD/9HS | 14CD/2HS | 141CD/97HS |
| Race | 11C/5NC | 42C/11NC | 5C/0NC | 43C/7NC | 45C/2NC | 14C/2NC | 192C/22NC |

CN = Cognitively normal, MCI = mild cognitive impairment, AD = Alzheimer's disease, N = number of subjects, MMSE = mini mental score examination, F = female and M = male.
Age is represented as mean ± standard deviation.
MMSE is also represented as mean ± standard deviation.
CD = College Degree or higher;
HS = High School or lower;
C = Caucasian;
NC = Asian, Black or African American or Other;
All the 109 subjects who received solanezumab were amyloid positive and mild to moderate AD cases

In a separate autopsy study, 67 subjects received ante-mortem flortaucipir scans and neuropathological findings were recorded according to NIA-AA recommendations by two experts.

TABLE 4
Demographics of the autopsy confirmed subjects.

| | B1 | | B2 | | B3 | |
|---|---|---|---|---|---|---|
| | Braak I | Braak II | Braak III | Braak IV | Braak V | Braak VI |
| N | 2 | 5 | 4 | 11 | 14 | 24 |
| Age | 67 ± 1.7 | 76.8 ± 6.8 | 85.8 ± 10.4 | 86.8 ± 6.3 | 85.6 ± 8.5 | 81.7 ± 9.4 |
| Sex | 1F/1M | 3F/2M | 3F/1M | 6F/5M | 5F/9M | 15F/9M |
| Education | 1CD/1HS | 2CD/3HS | 3CD/1HS | 4CD/7HS | 11CD/3HS | 14CD/10HS |
| Race | 2C/0NC | 4C/1NC | 4C/0NC | 11C/0NC | 14C/0NC | 24C/0NC |

N is the number of subjects, age is represented as mean ± S.D and gender is shown as female/male; CD = College Degree or higher; HS = High School or lower; C = Caucasian; NC = Asian, Black or African American or Other.

Two VOIs: Eτ comprising Freesurfer volumes (bilateral entorhinal cortex, fusiform, parahippocampal, and inferior temporal gyri) transformed to MNI space and previously published global AD-signature weighted neocortical region (MUBADA, also referred to as AD_{signature}) were used to calculate SUVR relative to a white matter reference region (PERSI). SUVR cutoffs for positivity were determined based on a cohort of young, cognitively normal subjects. Subjects were grouped based on the T+ positivity on both VOIs (Eτ+/AD-signature+; Eτ+/AD-signature−; Eτ−/AD-signature−). Groupwise comparisons were performed for baseline SUVR, 18-month changes in SUVR, neurodegeneration, and cognition. For the autopsy study, the sensitivity of Eτ in identifying intermediate Braak pathology (B2) subjects was compared to that of AD-signature− weighted neocortical VOI. The average surface maps of subjects in Eτ+/AD-signature− group and B2 NFT scores were created for visual evaluation of uptake.

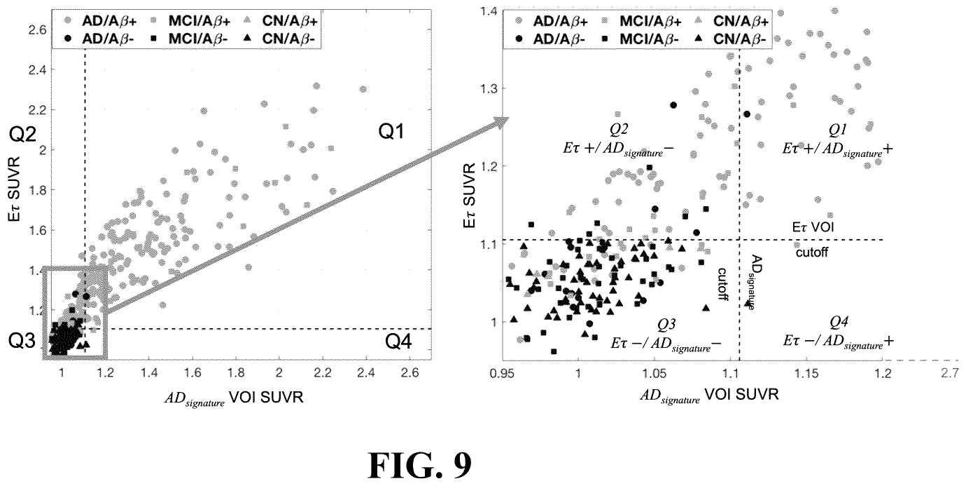

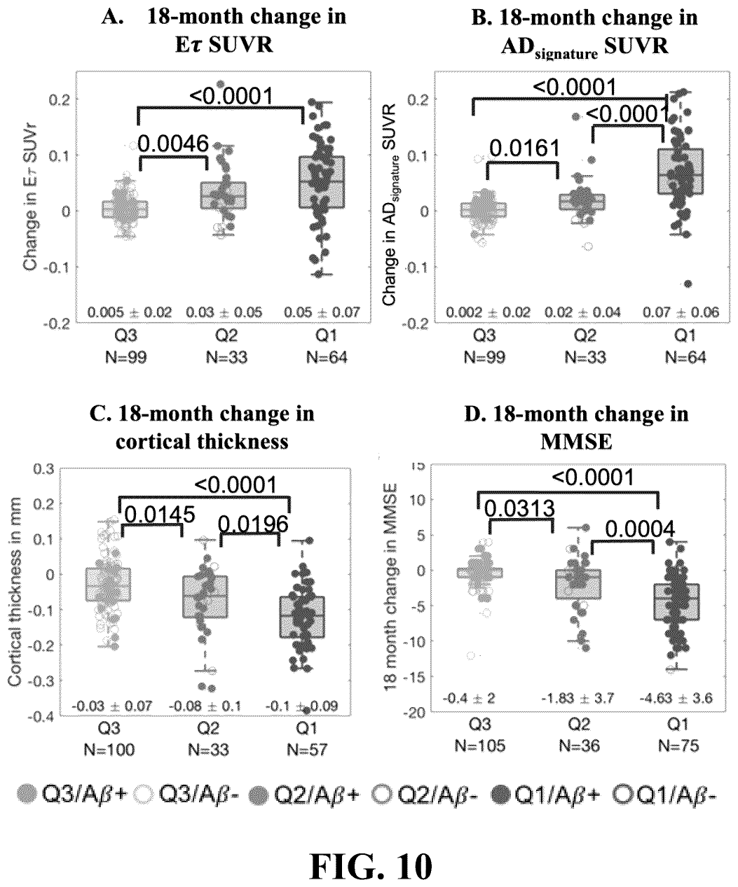

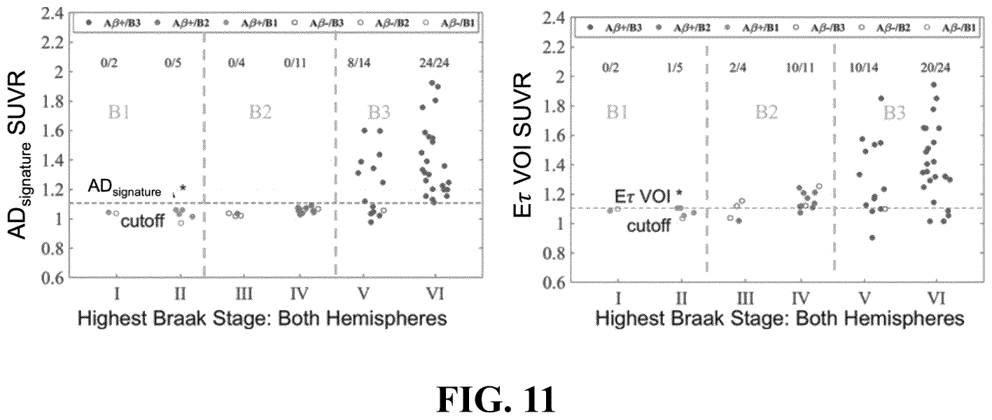

Results: Sixty four out of 390 analyzable subjects were identified as Eτ+/AD-signature−: 84% were Aβ+, 100% were diagnosed as MCI or AD and 59% were APOE E4 carriers. Subjects positive for both Eτ and MUBADA VOIs were represented in quadrant 1(Q1), subjects positive for Eτ alone were in quadrant 2 (Q2), subjects with low Eτ and MUBADA are in quadrant 3 (Q3), and subjects with low Eτ and high MUBADA in quadrant 4 (Q4) (FIG. 9). Since only two subjects were present in Q4, analysis was not performed on Q4. Consistent with the hypothesis that Eτ+/AD-signature− status reflects an early stage of AD, Eτ+/AD-signature− subjects deteriorated significantly faster than Eτ−/AD-signature− subjects, but significantly slower than Eτ+/AD-signature+ subjects, on most measures (i.e., change in AD-signature VOI SUVR, Eτ VOI, cortical thickness and MMSE) (FIG. 10). The AD-signature region was selective for subjects who came to autopsy with B3 (Braak V/VI) NFT score. In the autopsy study, 12/15 B2 subjects (including 10/11 Braak IV) were Eτ+/AD-signature− (FIG. 11) Surface maps showed that flortaucipir uptake was largely captured by the Eτ VOI regions in B2 subjects.

Summary: The Eτ VOI identified subjects with elevated temporal but not global tau (Eτ+/AD-signature−) that were primarily amyloid positive (Aβ+), APOE E4 carriers, and diagnosed as MCI or AD. Eτ+/AD-signature− subjects had greater accumulation of tau, greater atrophy, and higher decline on MMSE in 18-months compared to Eτ−/AD-signature− subjects. Finally, the Eτ VOI identified majority of the intermediate NFT score subjects in an autopsy-confirmed study. This study presents a visualization of ante-mortem FTP retention patterns that at a group level agree with the neurofibrillary tangle staging scheme proposed by Braak. These findings suggest that the Eτ VOI may be sensitive for detecting impaired subjects early in the course of Alzheimer's disease.

### Exemplified Embodiments of the Present Disclosure

The following provides embodiments set forth throughout the present disclosure.
1. A method of treating a patient having or suspected of having Alzheimer's disease, the method comprising: analyzing a tau-PET scan of a brain region of the patient to determine a tau-PET SUVR; and administering an Alzheimer's disease therapy to the patient if the tau-PET SUVR ranges from about 1.10 to about 1.45.
2. The embodiment of 1, wherein the Alzheimer's disease therapy is selected from donanemab, N3pG IV or LY3372993, LY3372689, ADUHELM®, solanezumab, gantenerumab and lecanemab.
3. The embodiment of any one of 1 or 2, wherein the brain region is inferior temporal brain region and the tau-PET SUVR is 1.45.
4. The embodiment of any one of 1 to 3, wherein the brain region is lateral temporal brain region and the tau-PET SUVR ranges from about 1.35 to about 1.45.
5. The embodiment of any one of 1 to 4, wherein the brain region is middle and superior temporal brain region and the tau-PET SUVR ranges from about 1.35 to about 1.45.
6. The embodiment of any one of 1 to 5, wherein the brain region is lateral parietal brain region and the tau-PET SUVR ranges from about 1.10 to about 1.45.
7. The embodiment of any one of 1 to 6, further comprising analyzing an amyloid-positron emission tomography (PET) scan to determine amyloid status.
8. The embodiment of any one of 1 to 7, further determining cortical thickness.
9. The embodiment of any one of 1 to 8, further comprising analyzing cerebrospinal fluid for amyloid-β.
10. The embodiment of any one of 1 to 9, further comprising analyzing epsilon-4 allele of apolipoprotein E (APOE E4) genotype.
11. The embodiment of any one of 1 to 10, further comprising obtaining a tau-positron emission tomography (PET) scan from a brain region of the patient and analyzing the tau-PET scan to determine a tau-PET SUVR following administration of the Alzheimer's disease therapy.
12. The embodiment of any one of 1 to 11, wherein the patient has or is suspected of having amyloid-plaques.
13. The embodiment of any one of 1 to 12, wherein the patient has or is suspected of having Alzheimer's disease cognitive decline.
14. A method of diagnosing a patient as having or suspected of having Alzheimer's disease, the method comprising: analyzing a tau-positron emission tomography (PET) scan from a brain region of the patient to determine a tau-PET SUVR; and diagnosing the patient as having or as suspected of having Alzheimer's disease if the tau-PET SUVR ranges from about 1.10 to about 1.45.
15. The embodiment of 14, wherein the brain region is inferior temporal brain region and the tau-PET SUVR is 1.45.
16. The embodiment of any one of 14 or 15, wherein the brain region is lateral temporal brain region and the tau-PET SUVR ranges from about 1.35 to about 1.45.
17. The embodiment of any one of 14 to 16, wherein the brain region is middle and superior temporal brain region and the tau-PET SUVR ranges from about 1.35 to about 1.45.
18. The embodiment of any one of 14 to 17, wherein the brain region is lateral parietal brain region and the tau-PET SUVR ranges from about 1.10 to about 1.45.
19. The embodiment of any one of 14 to 18, further comprising analyzing an amyloid-positron emission tomography (PET) scan to determine amyloid status.
20. The embodiment of any one of 14 to 19, further comprising determining cortical thickness.
21. The embodiment of any one of 14 to 20, further comprising analyzing cerebrospinal fluid for amyloid-β.
22. The embodiment of any one of 14 to 21, further comprising analyzing epsilon-4 allele of apolipoprotein E (APOE E4) genotype.
23. The embodiment of any one of 14 to 22, further comprising administering an Alzheimer's disease therapy to the patient if the tau-PET SUVR ranges from about 1.10 to about 1.45.
24. The embodiment of any one of 14 to 23, wherein the patient has or is suspected of having amyloid-plaques.
25. The embodiment of any one of 14 to 24, wherein the patient has or is suspected of having Alzheimer's disease cognitive decline.
26. The embodiment of any one of 14 to 25, further comprising administering an Alzheimer's disease therapy to the patient.
27. A method of identifying a patient having or suspected of having Alzheimer's disease as a candidate patient for receiving an Alzheimer's disease therapy, the method comprising: analyzing a tau-positron emission tomography (PET) scan from a brain region of the patient to determine a tau-PET SUVR; and identifying the patient as a candidate patient for receiving an Alzheimer's disease therapy if the tau-PET SUVR ranges from about 1.10 to about 1.45.
28. The embodiment of 27, wherein the brain region is inferior temporal brain region and the tau-PET SUVR is 1.45.
29. The embodiment of any one of 27 or 28, wherein the brain region is lateral temporal brain region and the tau-PET SUVR ranges from about 1.35 to about 1.45.
30. The embodiment of any one of 27 to 29, wherein the brain region is middle +superior temporal brain region and the tau-PET SUVR ranges from about 1.35 to about 1.45.
31. The embodiment of any one of 27 to 30, wherein the brain region is lateral parietal brain region and the tau-PET SUVR ranges from about 1.10 to about 1.45.
32. The embodiment of any one of 27 to 31, further comprising analyzing an amyloid-positron emission tomography (PET) scan to determine amyloid status.
33. The embodiment of any one of 27 to 32, further comprising determining cortical thickness.
34. The embodiment of any one of 27 to 33, further comprising analyzing cerebrospinal fluid for amyloid-β.
35. The embodiment of any one of 27 to 34, further comprising analyzing epsilon-4 allele of apolipoprotein E (APOE E4) genotype.
36. The embodiment of any one of 27 to 35, wherein the patient has or is suspected of having amyloid-plaques.
37. The embodiment of any one of 27 to 36, wherein the patient has or is suspected of having Alzheimer's disease cognitive decline.
38. The embodiment of any one of 27 to 38, further comprising administering an Alzheimer's disease therapy to the patient.
39. A method of treating a patient having or suspected of having amyloid plaques, the method comprising: analyzing a tau image of a brain region of the patient to determine a tau-PET SUVR; and administering an Alzheimer's disease therapy to the patient if the tau-PET SUVR ranges from about 1.10 to about 1.45.
40. The embodiment of 39, wherein the Alzheimer's disease therapy is selected from donanemab, LY3372689, N3pG IV or LY3372993, ADUHELM®, solanezumab, gantenerumab and lecanemab.
41. The embodiment of any one of 39 or 40, wherein the brain region is inferior temporal brain region and the tau-PET SUVR is 1.45.
42. The embodiment of any one of 39 to 41, wherein the brain region is lateral temporal brain region and the tau-PET SUVR ranges from about 1.35 to about 1.45.
43. The embodiment of any one of 39 to 42, wherein the brain region is middle and superior temporal brain region and the tau-PET SUVR ranges from about 1.35 to about 1.45.
44. The embodiment of any one of 39 to 43, wherein the brain region is lateral parietal brain region and the tau-PET SUVR ranges from about 1.10 to about 1.45.
45. The embodiment of any one of 39 to 44, further comprising analyzing an amyloid-positron emission tomography (PET) scan to determine amyloid status.
46. The embodiment of any one of 39 to 45, further determining cortical thickness.
47. The embodiment of any one of 39 to 46, further comprising analyzing cerebrospinal fluid for amyloid-β.
48. The embodiment of any one of 39 to 47, further comprising analyzing epsilon-4 allele of apolipoprotein E (APOE E4) genotype.
49. The embodiment of any one of 39 to 48, wherein the patient has or is suspected of having Alzheimer's disease cognitive decline.
50. The embodiment of any one of 39 to 49, further comprising obtaining a tau-positron emission tomography (PET) scan from a brain region of the patient and analyzing the tau-PET scan to determine a tau-PET SUVR following administration of the Alzheimer's disease therapy.
51. A method of diagnosing a patient as having or suspected of having amyloid plaques, the method comprising: analyzing a tau-positron emission tomography (PET) scan from a brain region of the patient to determine a tau-PET SUVR; and diagnosing the patient as having or as suspected of having amyloid plaques if the tau-PET SUVR ranges from about 1.10 to about 1.45.
52. The embodiment of 51, wherein the brain region is inferior temporal brain region and the tau-PET SUVR is 1.45.
53. The embodiment of 51 or 52, wherein the brain region is lateral temporal brain region and the tau-PET SUVR ranges from about 1.35 to about 1.45.
54. The embodiment of any one of 51 to 53, wherein the brain region is middle and superior temporal brain region and the tau-PET SUVR ranges from about 1.35 to about 1.45.
55. The embodiment of any one of 51 to 54, wherein the brain region is lateral parietal brain region and the tau-PET SUVR ranges from about 1.10 to about 1.45.
56. The embodiment of any one of 51 to 55, further comprising analyzing an amyloid-positron emission tomography (PET) scan to determine amyloid status.
57. The embodiment of any one of 51 to 56, further comprising determining cortical thickness.
58. The embodiment of any one of 51 to 57, further comprising analyzing cerebrospinal fluid for amyloid-β.
59. The embodiment of any one of 51 to 58, further comprising analyzing epsilon-4 allele of apolipoprotein E (APOE E4) genotype.
60. The embodiment of any one of 51 to 59, further comprising administering an Alzheimer's disease therapy to the patient if the tau-PET SUVR ranges from about 1.10 to about 1.45.
61. The embodiment of any one of 51 to 60, wherein the patient has or is suspected of having Alzheimer's disease cognitive decline.
62. The embodiment of any one of 51 to 60, further comprising obtaining a tau-positron emission tomography (PET) scan from a brain region of the patient and analyzing the tau-PET scan to determine a tau-PET SUVR following administration of the Alzheimer's disease therapy.
63. A method of identifying a patient having or suspected of having amyloid plaques as a candidate patient for receiving an Alzheimer's disease therapy, the method comprising: analyzing a tau-positron emission tomography (PET) scan from a brain region of the patient to determine a tau-PET SUVR; and identifying the patient as a candidate patient for receiving an Alzheimer's disease therapy if the tau-PET SUVR ranges from about 1.10 to about 1.45
64. The embodiment of 63, wherein the brain region is inferior temporal brain region and the tau-PET SUVR is 1.45.
65. The embodiment of any one of 63 or 64, wherein the brain region is lateral temporal brain region and the tau-PET SUVR ranges from about 1.35 to about 1.45.
66. The embodiment of any one of 63 or 64, wherein the brain region is middle+superior temporal brain region and the tau-PET SUVR ranges from about 1.35 to about 1.45.
67. The embodiment of any one of 63 to 66, wherein the brain region is lateral parietal brain region and the tau-PET SUVR ranges from about 1.10 to about 1.45.
68. The embodiment of any one of 63 to 67, further comprising analyzing an amyloid-positron emission tomography (PET) scan to determine amyloid status.
69. The embodiment of any one of 63 to 68, further comprising determining cortical thickness.
70. The embodiment of any one of 63 to 69, further comprising analyzing cerebrospinal fluid for amyloid-β.
71. The embodiment of any one of 63 to 70, further comprising analyzing epsilon-4 allele of apolipoprotein E (APOE E4) genotype.
72. The embodiment of any one of 63 to 71, wherein the patient has or is suspected of having Alzheimer's disease cognitive decline.
73. The embodiment of any one of 63 to 72, further comprising obtaining a tau-positron emission tomography (PET) scan from a brain region of the patient and analyzing the tau-PET scan to determine a tau-PET SUVR following administration of the Alzheimer's disease therapy.
74. A method of identifying cognitive decline in a patient having or suspected of having Alzheimer's disease, the method comprising: analyzing a tau-positron emission tomography (PET) scan from a brain region of the patient to determine a tau-PET SUVR; and identifying the patient as having cognitive decline if the tau-PET SUVR ranges from about 1.10 to about 1.45.
75. The embodiment of 74, wherein the brain region is inferior temporal brain region and the tau-PET SUVR is 1.45.
76. The embodiment of any one of 74 or 75, wherein the brain region is lateral temporal brain region and the tau-PET SUVR ranges from about 1.35 to about 1.45.
77. The embodiment of any one of 74 to 76, wherein the brain region is middle and superior temporal brain region and the tau-PET SUVR ranges from about 1.35 to about 1.45.
78. The embodiment of any one of 74 to 77, wherein the brain region is lateral parietal brain region and the tau-PET SUVR ranges from about 1.10 to about 1.45.
79. The embodiment of any one of 74 to 78, further comprising analyzing an amyloid-positron emission tomography (PET) scan to determine amyloid status.
80. The embodiment of any one of 74 to 79, further comprising determining cortical thickness.
81. The embodiment of any one of 74 to 80, further comprising analyzing cerebrospinal fluid for amyloid-β.
82. The embodiment of any one of 74 to 81, further comprising analyzing epsilon-4 allele of apolipoprotein E (APOE E4) genotype.
83. The embodiment of any one of 74 to 82, wherein the patient has or is suspected of having amyloid plaques.
84. The embodiment of any one of 74 to 83, further comprising obtaining a tau-positron emission tomography (PET) scan from a brain region of the patient and analyzing the tau-PET scan to determine a tau-PET SUVR following administration of the Alzheimer's disease therapy.
85. A method of identifying whether a patient receiving an Alzheimer's disease therapy is responding to the Alzheimer's disease therapy, the method comprising: analyzing a tau-positron emission tomography (PET) scan from a brain region of the patient to determine a tau-PET SUVR; administering an Alzheimer's disease therapy to the patient; analyzing a tau-positron emission tomography (PET) scan from the brain region of the patient to determine a tau-PET SUVR after administering the Alzheimer's disease therapy; and identifying the patient as responding to the Alzheimer's disease therapy if the tau-PET SUVR before administering the Alzheimer's disease therapy is changed based on determination of the tau-PET SUVR after administering the Alzheimer's disease therapy.
86. The embodiment of 85, wherein the brain region is inferior temporal brain region and the tau-PET SUVR after administering the Alzheimer's disease therapy is less than 1.45.
87. The embodiment any one of 85 or 86, wherein the brain region is lateral temporal brain region and the tau-PET SUVR after administering the Alzheimer's disease therapy is less than 1.35.
88. The embodiment any one of 85 to 87, wherein the brain region is middle and superior temporal brain region and the tau-PET SUVR after administering the Alzheimer's disease therapy is less than 1.35.
89. The embodiment any one of 85 to 88, wherein the brain region is lateral parietal brain region and the tau-PET SUVR after administering the Alzheimer's disease therapy is less than 1.10.
90. The embodiment any one of 85 to 89, wherein the patient has or is suspected of having amyloid plaques.
91. The embodiment any one of 85 to 90, wherein the patient has or is suspected of having Alzheimer's disease cognitive decline.
92. A method of determining tau burden in a patient having or suspected of having Alzheimer's disease, the method comprising: analyzing a tau-positron emission tomography (PET) scan from a brain region of the patient to determine a tau-PET SUVR; and identifying the patient as having tau burden if the tau-PET SUVR is greater than 1.15.
93. The embodiment of 92, wherein the brain region is inferior temporal brain region and the tau-PET SUVR is greater than 1.40.
94. The embodiment of any one of 92 or 93, wherein the brain region is lateral temporal brain region and the tau-PET SUVR is greater than 1.30.
95. The embodiment of any one of 92 to 94, wherein the brain region is middle and superior temporal brain region and the tau-PET SUVR is greater than 1.30.
96. The embodiment of any one of 92 to 95, wherein the brain region is lateral parietal brain region and the tau-PET SUVR is greater than 1.15.
97. The embodiment of any one of 92 to 96, wherein the patient has or is suspected of having amyloid plaques.
98. The embodiment of any one of 92 to 97, wherein the patient has or is suspected of having Alzheimer's disease cognitive decline.
99. The embodiment of any one of 92 to 98, further comprising administering an Alzheimer's disease therapy to the patient.
100. A method for determining whether a patient is a candidate for enrollment in an Alzheimer's disease clinical trial, the method comprising: analyzing a tau-positron emission tomography (PET) scan from a brain region of the patient to determine a region-based tau-PET SUVR; and identifying the patient as a candidate for enrollment if the tau-PET SUVR is greater than 1.15.
101. The embodiment of 100, wherein the brain region is inferior temporal brain region and the tau-PET SUVR is greater than 1.40.
102. The embodiment of any one of 100 or 101, wherein the brain region is lateral temporal brain region and the tau-PET SUVR is greater than 1.30.
103. The embodiment of any one of 100 to 102, wherein the brain region is middle and superior temporal brain region and the tau-PET SUVR is greater than 1.30.
104. The embodiment of any one of 100 to 103, wherein the brain region is lateral parietal brain region and the tau-PET SUVR is greater than 1.15.
105. The embodiment of any one of 100 to 104, wherein the patient has or is suspected of having amyloid plaques.
106. The embodiment of any one of 100 to 105, wherein the patient has or is suspected of having Alzheimer's disease cognitive decline.
107. The embodiment of any one of 100 to 106, further comprising administering an Alzheimer's disease therapy to the patient.
100. The embodiment of any one of 100 to 107, further comprising analyzing the patient for mild cognitive impairment, Alzheimer's disease, amyloid-beta, APOE E4 genotype, and combinations thereof.
101. The embodiment of any one of 100 to 108, further comprising determining tau in a plasma sample, cerebrospinal fluid, and combinations thereof from the patient.
110. A method of treating Alzheimer's disease in a patient that has been identified as amyloid positive comprising administering an Alzheimer's disease therapy, wherein the patient is identified as amyloid positive based on tau-PET scan.
111. The embodiment of 110, further comprising identifying a patient as at risk for cognitive decline by tau-PET scan.
112. The embodiment 110 or 111, wherein the Alzheimer's disease therapy is selected from donanemab, ADUHELM®, solanezumab, and lecanemab.
113. The embodiment any of 110-112, wherein the tau-PET scan examines the inferior temporal brain region and the tau-PET SUVR is 1.45.
114. The embodiment of any of 110-113, wherein the tau-PET scan examines the lateral temporal brain region and the tau-PET SUVR ranges from about 1.35 to about 1.45.
115. The embodiment of any of 110-114, wherein the tau-PET scan examines the middle and superior temporal brain region and the tau-PET SUVR ranges from about 1.35 to about 1.45.
116. The embodiment of any of 110-115, wherein the tau-PET scan examines the lateral parietal brain region and the tau-PET SUVR ranges from about 1.10 to about 1.45.
117. A method of treating Alzheimer's disease in a patient that has been identified as being at risk for Alzheimer's cognitive decline, comprising administering an Alzheimer's disease therapy, wherein the patient is identified as being at risk by tau-PET scan.
118. The embodiment of 117, further comprising identifying the patient as amyloid positive based on tau-PET scan.
119. The embodiment of 117 or 118, wherein the Alzheimer's disease therapy is selected from donanemab, LY3372689, N3pG IV or LY3372993, ADUHELM®, solanezumab, gantenerumab and lecanemab.
120. The embodiment of any of 117-119, wherein the tau-PET scan examines the inferior temporal brain region and the tau-PET SUVR is 1.45.
121. The embodiment of any of 117-120, wherein the tau-PET scan examines the lateral temporal brain region and the tau-PET SUVR ranges from about 1.35 to about 1.45.
122. The embodiment of any of 117-121, wherein the tau-PET scan examines the middle and superior temporal brain region and the tau-PET SUVR ranges from about 1.35 to about 1.45.
123. The embodiment of any of 117-122, wherein the tau-PET scan examines the lateral parietal brain region and the tau-PET SUVR ranges from about 1.10 to about 1.45.
124. A method of determining that a patient is at risk for cog decline comprising administering a tau-PET scan, wherein patient is determined as at risk based upon the SUVR level being in a certain brain region and/or at a certain level.
125. The embodiment of 124, further comprising determining whether the patient is amyloid positive based on the tau-PET scan.
126. The embodiment of 124 or 125, further comprising administering an Alzheimer's disease therapy to the patient, wherein the therapy is selected from donanemab, LY3372689, N3pG IV or LY3372993, ADUHELM®, solanezumab, gantenerumab and lecanemab.
127. The embodiment of any of 124-126, wherein the tau-PET scan examines the inferior temporal brain region and the tau-PET SUVR is 1.45.
128. The embodiment of any of 124-127, wherein the tau-PET scan examines the lateral temporal brain region and the tau-PET SUVR ranges from about 1.35 to about 1.45.
129. The embodiment of any of 124-128, wherein the tau-PET scan examines the middle and superior temporal brain region and the tau-PET SUVR ranges from about 1.35 to about 1.45.
130. The embodiment of any of 124-129, wherein the tau-PET scan examines the lateral parietal brain region and the tau-PET SUVR ranges from about 1.10 to about 1.45.
131. A method of treating Alzheimer's disease in a patient comprising identifying the patient as amyloid positive based on a tau-PET scan; and administering a therapy to the patient when the tau-PET scan is above a threshold.
132. The embodiment of 131, further comprising identifying that the patient as at risk for Alzheimer's cognitive decline based upon the tau-PET scan.
133. The embodiment of any of 131-132, wherein the therapy is selected from donanemab, LY3372689, N3pG IV or LY3372993, ADUHELM®, solanezumab, gantenerumab and lecanemab.
134. The embodiment of any of 131-133, wherein the tau-PET scan examines the inferior temporal brain region and the threshold is a tau-PET SUVR of 1.45.
135. The embodiment of any of 131-134, wherein the tau-PET scan examines the lateral temporal brain region and the threshold is a tau-PET SUVR ranging from about 1.35 to about 1.45.
136. The embodiment of any of 131-135, wherein the tau-PET scan examines the middle and superior temporal brain region and the threshold is a tau-PET SUVR ranging from about 1.35 to about 1.45.
137. The embodiment of any of 131-136, wherein the tau-PET scan examines the lateral parietal brain region and the =threshold is a tau-PET SUVR ranging from about 1.10 to about 1.45.
138. A method of treating Alzheimer's disease in a patient comprising identifying that the patient as at risk for Alzheimer's cognitive decline based upon the tau-PET scan; and administering a therapy to the patient when the tau-PET scan is above a threshold.
139. The embodiment of 138, further comprising identifying the patient as amyloid positive based on a tau-PET scan.
140. The embodiment of any of 138-139, wherein the therapy is selected from donanemab, LY3372689, N3pG IV or LY3372993, ADUHELM®, solanezumab, gantenerumab and lecanemab.
141. The embodiment of any of 138-140, wherein the tau-PET scan examines the inferior temporal brain region and the threshold is a tau-PET SUVR of 1.45.
142. The embodiment of any of 138-141, wherein the tau-PET scan examines the lateral temporal brain region and the threshold is a tau-PET SUVR ranging from about 1.35 to about 1.45.
143. The embodiment of any of 138-142, wherein the tau-PET scan examines the middle and superior temporal brain region and the threshold is a tau-PET SUVR ranging from about 1.35 to about 1.45.
144. The embodiment of any of 138-143, wherein the tau-PET scan examines the lateral parietal brain region and the threshold is a tau-PET SUVR ranging from about 1.10 to about 1.45.
145. A method of determining that a patient is amyloid positive comprising administering a tau-PET scan, wherein patient is determined as being amyloid positive if the tau-PET scan is above a certain threshold.
146. The embodiment of 145, wherein the tau-PET scan examines the inferior temporal brain region and the threshold is a tau-PET SUVR of 1.45.
147. The embodiment of any of 145-146, wherein the tau-PET scan examines the lateral temporal brain region and the threshold is a tau-PET SUVR ranging from about 1.35 to about 1.45.
148. The embodiment of any of 145-147, wherein the tau-PET scan examines the middle and superior temporal brain region and the threshold is a tau-PET SUVR ranging from about 1.35 to about 1.45.
149. The embodiment of any of 145-148, wherein the tau-PET scan examines the lateral parietal brain region and the threshold is a tau-PET SUVR ranging from about 1.10 to about 1.45.
150. The embodiment of any of 145-149, further comprising determining that the patient is at risk for Alzheimer's cognitive decline based upon the tau-PET scan being above the threshold.
151. A method of determining that a patient is at risk for Alzheimer's cognitive decline comprising administering a tau-PET scan, wherein patient is determined to be at risk for Alzheimer's cognitive decline if the tau-PET scan is above a certain threshold.
152. The embodiment of 151, wherein the tau-PET scan examines the inferior temporal brain region and the threshold is a tau-PET SUVR of 1.45.
153. The embodiment of any of 151-152, wherein the tau-PET scan examines the lateral temporal brain region and the threshold is a tau-PET SUVR ranging from about 1.35 to about 1.45.
154. The embodiment of any of 151-153, wherein the tau-PET scan examines the middle and superior temporal brain region and the threshold is a tau-PET SUVR ranging from about 1.35 to about 1.45.
155. The embodiment of any of 151-154, wherein the tau-PET scan examines the lateral parietal brain region and the threshold is a tau-PET SUVR ranging from about 1.10 to about 1.45.
156. The embodiment of any of 151-155, further comprising determining that the patient is amyloid positive, wherein patient is determined as being amyloid positive if the tau-PET scan is above the certain threshold.
157. A method of treating Alzheimer's disease in a patient identified as being amyloid positive comprising administering an Alzheimer's therapy, wherein the patient identified as being amyloid positive based on a tau-PET scan.
The embodiment of 157, wherein the tau-PET scan examines the inferior temporal brain region and the tau-PET scan identifies the patient if the tau-PET SUVR is 1.45.
159. The embodiment of any of 157-158, wherein the tau-PET scan examines the lateral temporal brain region and the tau-PET scan identifies the patient if the tau-PET SUVR ranges from about 1.35 to about 1.45.
160. The embodiment of any of 157-159, wherein the tau-PET scan examines the middle and superior temporal brain region and region and the tau-PET scan identifies the patient if the tau-PET SUVR ranges from about 1.35 to about 1.45.
161. The embodiment of any of 157-160, wherein the tau-PET scan examines the lateral parietal brain region and region and the tau-PET scan identifies the patient if the tau-PET SUVR ranges from about 1.10 to about 1.45.
162. The embodiment of any of 157-161, further wherein the Alzheimer's therapy is selected from donanemab, LY3372689, N3pG IV or LY3372993, ADUHELM®, solanezumab, gantenerumab and lecanemab.
163. A method of treating Alzheimer's disease in a patient identified as being at risk for Alzheimer's cognitive decline comprising administering an Alzheimer's therapy, wherein the patient identified as being at risk for Alzheimer's cognitive decline based on a tau-PET scan.
164. The embodiment of 163, wherein the tau-PET scan examines the inferior temporal brain region and the tau-PET scan identifies the patient if the tau-PET SUVR is 1.45.
165. The embodiment of any of 163-164, wherein the tau-PET scan examines the lateral temporal brain region and the tau-PET scan identifies the patient if the tau-PET SUVR ranges from about 1.35 to about 1.45.
166. The embodiment of any of 163-165, wherein the tau-PET scan examines the middle and superior temporal brain region and region and the tau-PET scan identifies the patient if the tau-PET SUVR ranges from about 1.35 to about 1.45.
167. The embodiment of any of 163-166, wherein the tau-PET scan examines the lateral parietal brain region and region and the tau-PET scan identifies the patient if the tau-PET SUVR ranges from about 1.10 to about 1.45.
168. The embodiment of any of 163-167, further comprising determining that the patient is amyloid positive, wherein patient is determined as being amyloid positive if the tau-PET scan is above the certain threshold.
169. The embodiment of any of 163-168, further wherein the Alzheimer's therapy is selected from donanemab, LY3372689, N3pG IV or LY3372993, ADUHELM®, solanezumab, gantenerumab and lecanemab.
170. A method of treating Alzheimer's disease in a patient identified as being amyloid positive and identified as being at risk for Alzheimer's cognitive decline comprising administering an Alzheimer's therapy, wherein the patient identified as being amyloid positive and identified as being at risk for Alzheimer's cognitive decline based on a tau-PET scan.
171. The embodiment of 170, wherein the tau-PET scan examines the inferior temporal brain region and the tau-PET scan identifies the patient if the tau-PET SUVR is 1.45.
172. The embodiment of any of 170-171, wherein the tau-PET scan examines the lateral temporal brain region and the tau-PET scan identifies the patient if the tau-PET SUVR ranges from about 1.35 to about 1.45.
173. The embodiment of any of 170-172, wherein the tau-PET scan examines the middle and superior temporal brain region and region and the tau-PET scan identifies the patient if the tau-PET SUVR ranges from about 1.35 to about 1.45.
174. The embodiment of any of 170-173, wherein the tau-PET scan examines the lateral parietal brain region and region and the tau-PET scan identifies the patient if the tau-PET SUVR ranges from about 1.10 to about 1.45.
175. The of any of 170-174, further wherein the Alzheimer's therapy is selected from donanemab, LY3372689, N3pG IV or LY3372993, ADUHELM®, solanezumab, gantenerumab and lecanemab.
176. The embodiment of any of 157-162, wherein the patient is further identified as being at risk for Alzheimer's cognitive decline based on a tau-PET scan.

In view of the above, it will be seen that the advantages of the disclosure are achieved, and other advantageous results attained. As various changes could be made in the above methods without departing from the scope of the disclosure, it is intended that all matter contained in the above description and shown in the accompanying drawings shall be interpreted as illustrative and not in a limiting sense.

When introducing elements of the present disclosure or the various versions, embodiment(s) or aspects thereof, the articles “a”, “an”, “the” and “said” are intended to mean that there are one or more of the elements. The terms “comprising”, “including” and “having” are intended to be inclusive and mean that there may be additional elements other than the listed elements.

1. A method of identifying a patient having or suspected of having Alzheimer's disease as a candidate patient for receiving an Alzheimer's disease therapy, the method comprising: analyzing a tau-positron emission tomography (PET) scan from a brain region of the patient to determine a tau-PET SUVR; and identifying the patient as a candidate patient for receiving an Alzheimer's disease therapy if the tau-PET SUVR ranges from about 1.05 to about 1.45.

2. The method of claim 1, wherein the brain region is the inferior temporal brain region.

3. The method of claim 1, wherein the brain region is the lateral temporal brain region and the tau-PET SUVR ranges from about 1.35 to about 1.45.

4. The method of claim 1, wherein the brain region is the middle and superior temporal brain region and the tau-PET SUVR ranges from about 1.35 to about 1.45.

5. The method of claim 1, wherein the brain region is the lateral parietal brain region and the tau-PET SUVR ranges from about 1.10 to about 1.45.

6. The method of claim 1, wherein the brain region is the bilateral entorhinal cortex brain region and the tau-PET SUVR ranges from about 1.05 to about 1.45.

7. The method of claim 1, wherein the brain region is the fusiform brain region and the tau-PET SUVR ranges from about 1.05 to about 1.45.

8. The method of claim 1, wherein the brain region is the parahippocampal brain region and the tau-PET SUVR ranges from about 1.05 to about 1.45.

9. The method of claim 1, wherein the brain region is the inferior temporal brain region and the tau-PET SUVR is about 1.45.

10. The method of claim 1, further comprising analyzing an amyloid-positron emission tomography (PET) scan to determine amyloid status.

11. The method of claim 1, further comprising determining cortical thickness.

12. The method of claim 1, further comprising analyzing cerebrospinal fluid for amyloid-β.

13. The method of claim 1, further comprising analyzing epsilon-4 allele of apolipoprotein E (APOE ε4) genotype.

14. The method of claim 10, wherein the patient is identified as having amyloid plaques and/or is at a risk for developing amyloid plaques based upon the tau-PET SUVR.

15. The method of claim 1, wherein the patient is identified as being at a risk for having Alzheimer's disease cognitive decline based upon the tau-PET SUVR.

16. The method of claim 15, further comprising administering an Alzheimer's disease therapy to the patient.

17. A method of treating a patient having or suspected of having Alzheimer's disease, the method comprising: analyzing a tau-PET scan of a brain region of the patient to determine a tau-PET SUVR; and administering an Alzheimer's disease therapy to the patient if the tau-PET SUVR ranges from about 1.05 to about 1.45.

18. A method of treating a patient identified as having or determined as having amyloid plaques, the method comprising: analyzing a tau image of a brain region of the patient to determine a tau-PET SUVR; and administering an Alzheimer's disease therapy to the patient if the tau-PET SUVR ranges from about 1.05 to about 1.45.

19. The method of claim 16, wherein the Alzheimer's disease therapy is donanemab, LY3372689, N3pG IV or LY3372993, ADUHELM®, solanezumab, gantenerumab, or lecanemab.

20.-36. (canceled)

37. A method of diagnosing a patient as having or suspected of having Alzheimer's disease, the method comprising: analyzing a tau-positron emission tomography (PET) scan from a brain region of the patient to determine a tau-PET SUVR; and
diagnosing the patient as having or as suspected of having Alzheimer's disease if the tau-PET SUVR ranges from about 1.05 to about 1.45.

38.-53. (canceled)
